(19) United States
(12) Patent Application Publication
(10) Pub. No.: US 2019/0375842 A1
(43) Pub. Date: Dec. 12, 2019

(54) CHECK POINT INHIBITION IN ORGAN FIBROSIS

(71) Applicant: VANDERBILT UNIVERSITY, Nashville, TN (US)

(72) Inventor: Wonder P. Drake, Joelton, TN (US)

(21) Appl. No.: 16/240,462

(22) Filed: Jan. 4, 2019

Related U.S. Application Data
(60) Provisional application No. 62/613,600, filed on Jan. 4, 2018.

(51) Int. Cl. C07K 16/28; G01N 33/68

(52) U.S. Cl. CPC C07K 16/2818; A61K 45/06; G01N 33/6869; G01N 33/6893

(57) ABSTRACT

Disclosed are compositions and methods for treating pulmonary fibrosis with PD1 blockade, STAT3 inhibitors, IL-17 inhibitors, and/or TGFβ-1 inhibitors.

This application claims the benefit of U.S. Provisional Application No. 62/613,600, filed on Jan. 4, 2018 which is incorporated herein by reference in its entirety.

### I. BACKGROUND

Pulmonary fibrosis represents progressive remodeling of lung architecture by deposition of connective tissue elements following persistent stimulation from antigenic (Schistosoma eggs) and non-antigenic sources (bleomycin). While tissue repair in its early stages are beneficial, continued connective tissue deposition results in scar formation that eventually leads to organ fibrosis and death without therapeutic interventions, such as antifibrotic agents. While new therapies continue to emerge, enhanced understanding of the molecular foundation for immunologic mediation of pulmonary fibrosis increases the opportunity to provide therapy that directly targets pathophysiology. What are needed are new therapies to treat pulmonary fibrosis and related conditions.

### II. SUMMARY

Disclosed are methods and compositions related to treating a pulmonary disease (for example, an interstitial lung disease such as idiopathic pulmonary fibrosis, bleomycin-induced pulmonary fibrosis, or sarcoidosis) in a subject comprising administering to the subject an agent that reduces STAT3 production or activation and/or inhibitor IL-17A and/or TGFβ-1.

In one aspect, disclosed herein are methods and compositions of treating a pulmonary disease comprising administering to a subject with a pulmonary disease an agent, wherein the agent is a small molecule, peptide, polypeptide, siRNA, or antibody.

Also disclosed herein are methods of treating a pulmonary disease of any preceding aspect, wherein the agent inhibits the interaction of PD-1 and PDL-1 (such as, for example, an anti-PD1 antibody), inhibits the proliferation of T_{H}17 cells, or comprises a STAT3 inhibitor.

In one aspect, disclosed herein are methods of treating a pulmonary disease of any preceding aspect, wherein the STAT3 inhibitor is selected from the group consisting of Tyrosine phosphorylation inhibitors (Staitic, JSI-124, Withacnistin), SH2 domain inhibitors or dimerizaton inhibitors (PpYLKTK, SS 610, S31-201, S31-M2001, STA-21), and/or DNA binding domain inhibitors (IS3 295, CPA-1, CPA-7, Galielllalactone).

In one aspect, disclosed herein are methods of creating a prognosis for a subject with a pulmonary disease comprising obtaining a tissue sample form a subject, isolating the TGFβ+PD1+CD4 T cells (T_{H}17 cells), measuring the number of T_{H}17 CD4+ T cells, wherein an increase in TGFβ+PD1+T_{H}17 cells relative to a control and/or a decrease in IFNγ+IL-17A+T_{H}17 cells relative to a control indicates decreased survival chances for the subject. Also disclosed herein are methods of creating a prognosis for a subject with a pulmonary disease (such as, for example, an interstitial lung disease such as idiopathic pulmonary fibrosis, bleomycin-induced pulmonary fibrosis, or sarcoidosis) comprising obtaining a tissue sample form a subject, isolating the TGFβ+PD1+CD4 T cells (T_{H}17 cells), measuring the IL-17 expression levels relative to a control, wherein an increase in IL-17 expression levels relative to a control indicates a more aggressive disease and decreased survival chances for the subject.

In one aspect, disclosed herein are methods of creating a prognosis of any preceding aspect, wherein the T_{H}17 or Il-17 expression is measured by microarray, flow cytometry, or ELISA.

Also disclosed herein are methods of creating a prognosis for a subject with a pulmonary disease comprising obtaining a tissue sample from a subject, isolating the TGFβ+PD1+CD4 T cells (T_{H}17 cells), assaying for single nucleotide polymorphisms (SNPs) in the STAT3 gene (such as, for example, rs11079042, rs17881621, and/or rs80350541), IL-17RA gene (such as, for example, rs41482444), IL-17D gene (such as, for example, rs182051094 and/or rs41480348), ERAP2 gene (such as, for example, rs61731306, rs873723, and/or rs1047591), PDCD1 gene (such as, for example, rs143359677), and/or TGF-β1 gene (such as, for example, rs4669), wherein the presence of an assayed SNP in the STAT3 gene, IL-17RA gene, PDCD1 gene, or TGF-β1 gene indicate an increased risk of disease progression, the presence of a assayed SNP in the IL-17D gene indicates increased risk of persistence, and the presence of an assayed SNP in the ERAP2 gene indicates increased risk of disease progression and persistence.

Also disclosed herein are methods of creating a prognosis for a subject with a pulmonary disease comprising assaying for SNPs of any preceding aspect, wherein the SNP is identified by allele-specific PCR, sequencing, single base extension (SBE), oligonucleotide ligation assay (OLA), Heated oligonucleotide ligation assay (HOLA), mass spectrometry, single-strand conformation polymophism, and/or electrophoresis.

In one aspect, disclosed herein are methods of reducing collagen-1 production in a subject with a pulmonary disease comprising administering to the subject an agent that reduces STAT3 production or activation.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A and 1B show genome-wide Th17 gene expression profiles in IPF. (A) Heat map demonstrating differential expression of Th17-related genes in patients with IPF. (B) Percent transplant free survival for cluster 1 and cluster 2 over the course of 3.5 years post-diagnosis.

FIGS. 2A, 2B, 2C, 2D, 2E, 2F, 2G, 2H, 2I, and 2J show PD-1 upregulation in fibrotic lung disease is associated with loss of lung function. Gating strategy and representative histograms depicting (A) PD-1 levels and (B) proliferative capacity for a healthy control, a sarcoidosis resolver, progressor, and IPF patient. (C) Total peripheral CD4+ T cells expressing PD-1 from healthy control subjects (HC, n=30) and IPF patients (n=12). (D) Age-matched healthy controls and IPF CD4+ T cells that are PD-1+(HC, n=6; IPF, n=12). (E) Baseline percentages of CD4+PD-1+ T cells from peripheral blood mononuclear cells (PBMC) for HC (n=24), patients with sarcoidosis (n=36) and IPF (n=25). (F) Percent proliferation for CD4+ T cells from the peripheral blood of HC, (n=14), sarcoidosis (n=19) and IPF subjects (n=21) following TCR stimulation with plate-bound CD3/CD28 antibodies. (G) CD4+PD-1+ T cells (HC, n=24; Resolvers, n=9; Progressors, n=19; IPF, n=25) and (H) proliferating CD4+ T cell (HC, n=14; Resolvers, n=5; Progressors, n=12; IPF, n=21) comparisons between sarcoidosis and IPF cohorts according to disease state and immune function. Percent PD-1 expression amongst CD4+ T cell subsets from (I) sarcoidosis and (J) IPF patients. *p<0.05, **p<0.01, ***p<0.001, ****p<00001, NS=no significance.

FIGS. 3A, 3B, 3C, 3D, and 3E show bleomycin-induced lung fibrosis murine model reveals CD4+ T cells with increased PD-1 and reduced proliferative capacity. CD4+ T cell gating strategy from single cell lung suspensions from PBS- and bleomycin-treated C57BL/6J mice. (A) Increased CD4+PD-1+ T cell percentages and (B) MFI in mice 21 days post-bleomycin treatment. Histograms depicting percent PD-1 on CD4+ T cells that have not been TCR stimulated. (C) Percent and (D) MFI for CD4+PD-1+ T cells following TCR stimulation with plate-bound anti-CD3/CD28 antibodies (PBS, n=4; Bleomycin n=8). Representative histograms depicting percent PD-1 on CD4+ T cells following TCR stimulation. (E) Percent CD4+ T cell proliferation subsequent to 5-day TCR stimulation. Representative histograms illustrating percent of proliferating CD4+ T cells (CFSE negative) for a PBS- and bleomycin-treated mouse after TCR activation (PBS, n=3; bleomycin, n=5). *p<0.05, **p<0.01, ***p<0.001.

FIG. 4 shows that normal and IPF human lung biopsies demonstrate increased PD-1 and PD-L1 expression Immunohistochemistry assessment for PD-1 and PDL1 in healthy and IPF pulmonary biopsies. While scant expression of PD-1 or PD-L1 is noted on healthy control biopsies, IPF biopsies demonstrate increased PD-1 expression on epithelial cells, as well as lymphocytes in proximity PD-L1 expression is also noted within IPF biopsies.

FIGS. 5A, 5B, 5C, 5D, 5E, 5F, 5G, 5H, 5I, 5J, 5K, 5L, and 5M show that Th17 cells display the largest percentage of cells expressing TGF-β1. Peripheral CD4+ T cells from HC, sarcoidosis and IPF patients were TCR stimulated for 24 hours at 37° C. in 5% CO2 atmosphere. (A) FACS plots depicting TGF-β1 secretion for total CD4+ T cells, Tregs and Th17. (B) Percent and MFI for total CD4+ T cells expressing TGF-β1 from sarcoidosis and IPF subjects (HC, n=12; sarc, n=14; IPF, n=7). (C) Increased activated TGF-β1 released by sarcoidosis CD4+ T cells (HC, n=4; sarcoidosis, n=6). (D) PD-1 percentages for total CD4+ T cells expressing TGF-β1 (HC, n=6; sarc, n=10; IPF, n=7). Representative histograms for a HC, sarcoidosis and IPF patient portraying PD-1 percentages on cells that are positive for TGF-β1 secretion. (E) Percentile of T regulatory cells (T regs) that are TGF-β1+ in sarcoidosis and IPF patients compared to HC (HC, n=14; sarc, n=13; IPF, n=7) and (F) PD-1 expression on T regs that are positive for TGF-β1 (HC, n=8; sarc, n=10; IPF, n=7). Histograms depicting percent LAP/TGF-β1+PD-1+T regs. (G) Th17 cells expressing TGF-β1 (percent and MFI) in HC (n=6) and sarcoidosis patients according to baseline PD-1 levels (high, n=8; low, n=5) and in (H) IPF subjects (n=7). (I) Percent of Th17 cells that are positive for TGF-β1 and expressing PD-1 in patients with sarcoidosis and IPF (HC, n=6; sarc, n=13; IPF, n=7). Histograms depicting PD-1 levels on TGF-β1+Th17 cells for a healthy control, sarcoidosis and IPF patient. Single cell lung suspensions from PBS- and bleomycin-treated mice were anti-CD3/CD28 stimulated for 24 hours at 37° C. in 5% CO2 atmosphere. (J) Percent of Th17 cells expressing TGF-β1 (PBS, n=4; bleomycin, n=8). FACS plots illustrating TGF-β1 levels in PBS- and bleomycin-treated mice. (K) TGF-β1 percentages expressed T regs in PBS- (n=4) and bleomycin-treated (n=8) mice. Histograms representing percent TGF-β1 expressed by T regs. (L) CD4+ T cells subsets secreting TGF-β1 in sarcoidosis, IPF and bleomycin-treated mice. (M) Percent TGF-β1 expressed by each CD4+ T cell subset in each cohort.

FIGS. 6A, 6B, 6C, 6D, and 6E show that culturing of CD4+ T cells with human lung fibroblasts increases collagen I production. Human lung fibroblasts (HLF) were co-cultured with purified CD4+ T cells for 5 days at 37° C. in 5% CO2 atmosphere following TCR stimulation with plate-bound anti-CD3 and CD28 antibodies. (A) Fibroblast (CD45 negative) and fibrocyte (CD45 positive) gating strategy and representative histograms depicting collagen I levels for HLF cultured alone or with purified CD4+ T cells from healthy controls, sarcoidosis, or IPF patients. (B) Percent of fibroblasts (CD45-) expressing collagen I when cultured alone (n=14), and when cultured with HC (n=8) or sarcoidosis (n=12) CD4+ T cells with following TCR activation. (C) Collagen-I expression by fibroblasts when cultured with purified CD4+ T cells from IPF patients (n=4). (D) Percent of fibrocytes (CD45+) producing collagen when cultured alone (HLF) or with purified CD4+ T cells from healthy controls and sarcoidosis patients (HLF, n=14; HC, n=8; sarc, n=10). (E) Fibrocytes that are positive for collagen I when cultured with IPF CD4+ T cells (n=4). *p<0.05, **p<0.01, ***p<0.001, NS=no significance.

FIGS. 7A, 7B, 7C, 7D, 7E,7F, and 7G show that PD-1 blockade significantly decreases human lung fibroblast collagen 1 production. Purified sarcoidosis CD4+ T cells from the same patients were cultured with or without the presence of PD-1 blockade antibodies overnight, HLF were added the following day and the cells were cultured for 5 days at 37° C. in 5% CO2 atmosphere following TCR stimulation. (A) Representative histograms depicting collagen I expression for HLF alone or when cultured in combination with sarcoidosis CD4+ T cells pre- and post-PD-1 pathway blockade. (B) Percent of fibroblasts (CD45-) producing collagen I subsequent to PD-1 blockade (HLF, n=14; pre-blockade, n=19; post-blockade, n=13). Collagen I percentages expressed by fibroblasts following culturing with purified sarcoidosis CD4+ T cells that expressed (C) high or (D) low baseline PD-1 levels, pre- and post-blockade (high, n=9; low, n=4). (E) Percent of fibrocytes producing collagen I when cultured with CD4+ T cells from sarcoidosis patients prior to and following PD-1 blockade (HLF, n=14; pre-blockade, n=13; post-blockade, n=13). (F-G) Fibrocytes producing collagen I when cultured with sarcoidosis CD4+ T cells pre- and post-PD-1 pathway blockade according to baseline PD-1 (high PD-1, n=9; low, n=4). *p<0.05, **p<0.01, ***p<0.001, ****p<00001, NS=no significance.

FIGS. 8A, 8B, 8C, 8D, 8E, 8F, 8G, and 8H show that PD-1 blockade reduces IL-17A and TGF-β1 secretion in CD4+Th17 cells. Purified CD4+ T cells from the same sarcoidosis or IPF patients were either cultured alone or in the presence of HLF at 37° C. in 5% CO2 atmosphere following TCR stimulation with anti-CD3 and CD28 antibodies. (A-B) Increased CD4+IL-17A+ T cell percentages (n=4). FACS plots illustrating percent CD4+IL-17A+ T cells. (C) Isolated CD4+ T cells were cultured overnight with or without the presence of anti-PD-1, PD-L1 and PD-L2. The following day, cells were TCR stimulated and co-cultured with human lung fibroblasts/fibrocytes (HLF). Reduced CD4+IL-17A+ T cells following PD-1 blockade (n=6). Representative FACS plots illustrating CD4+IL-17A T cell percentages pre- and post-blockade. (D-E) Subsequent to PD-1 blockade, purified CD4+ T cells were TCR stimulated for 24 hours at 37° C. in 5% CO2. Percent of CD4+ T cells from sarcoidosis and IPF patients secreting TGF-β1 pre- and post-blockade (sarcoidosis, n=11; IPF, n=7). Representative FACS plots portraying percent TGF-β1 expressed by sarcoidosis and IPF CD4+ T cells pre- and post-PD-1 blockade. (F-G) Sarcoidosis Th17 cells expressing TGF-β1 following PD-1 pathway blockade according to CD4+ T cell baseline PD-1 levels (high PD-1, n=8; low PD-1, n=5). FACS plots depicting TGF-β1 levels for a sarcoidosis patient with high baseline PD-1 pre- and post-blockade and a sarcoidosis patient with low baseline PD-1 prior to and following blockade. (H) Percent of Th17 cells that are TGF-β1+ pre- and post-PD-1 blockade in IPF patients (n=7). Representative FACS plots showing percent Th17 cells that are expressing TGF-β1 pre- and post-blockade for an IPF subject. *p<0.05, **p<0.01, ***p<0.001, NS=no significance.

FIGS. 9A, 9B, 9C, 9D, 9E, 9F, 9G, and 9H show that STAT3 inhibition reduces collagen I production. Isolated CD4+ T cells from healthy control (HC) and sarcoidosis PBMC were anti-CD3 and anti-CD28 stimulated for 24 hours at 37° C. in 5% CO2. STAT3 expression was then assessed using flow cytometry. (A) Representative histograms depicting STAT3 expression for a healthy control and sarcoidosis subject. (B) Percent and (C) MFI of CD4+ T cells expressing STAT3 (HC, n=4; sarcoidosis, n=8). Purified CD4+ T cells were cultured overnight with or without the STAT3 inhibitor STATTIC. The following day, the cells were TCR stimulated in the presence of HLF and cultured at 37° C. in 5% CO2. STAT3 gene expression was assessed using quantitative RT-PCR. (D) STAT3 upregulation in sarcoidosis CD4+ T cells with high PD-1 baseline levels (HC, n=3; PD-1 hi, n=3; PD-1 low, n=6). (E) STAT3 expression restored following PD-1 pathway blockade (HC, n=3; pre-blockade, n=3; post-blockade, n=3). (F) Percent collagen I produced by fibroblasts (CD45-) that have been cultured in the presence of sarcoidosis CD4+ T cells without Stattic (orange bar) and with Stattic (green bar). (G) Percent of collagen I-producing fibrocytes (CD45+) following STAT3 inhibition in purified sarcoidosis CD4+ T cells. (H) Histograms illustrating percent collagen produced by fibroblasts (CD45-) and fibrocytes (CD45+) prior to STAT3 inhibition (orange) and following STAT3 inhibition (green) using STATTIC. *p<0.05, **p<0.01, ***p<0.001, NS=no significance.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

As the knowledge of the immunological events that play a role in pulmonary fibrosis increases, an opportunity emerges to provide therapy that directly targets pathophysiology through immunologic mediation of pulmonary fibrosis. Accordingly, in one aspect, disclosed herein are methods of treating a pulmonary disease in a subject.

The treatments disclosed herein are designed to mediate fibrosis associated with pulmonary disease. Thus, as used herein, pulmonary disease can be an interstitial lung disease. Interstitial lung diseases are a group of diseases associated with excessive fibrotic tissue in alveolar epithelium, pulmonary capillary endothelium, basement membrane, perivascular and perilymphatic tissues following an initial injury to the lungs. This makes it more difficult for oxygen to pass into the bloodstream. Accordingly, in one aspect, disclosed herein are methods of treating a pulmonary disease in a subject; wherein the pulmonary disease is an interstitial lung disease such as idiopathic pulmonary fibrosis, bleomycin-induced pulmonary fibrosis, or sarcoidosis) in a subject.

By “treat” or other forms of the word, such as “treated” or “treatment,” is meant to administer a composition or to perform a method in order to reduce, prevent, inhibit, or eliminate a particular characteristic or event (e.g., tumor growth or survival). The term “control” is used synonymously with the term “treat.” It is understood and herein contemplated that a treatment does not have to result in the ablation of the disease, but rather can be any reduction in the severity of the disease relative to a prior state in the subject with the disease or relative to an untreated control. Moreover, the treatment can result in reduction of symptoms of the disease rather than a reduction in the causative condition itself. For example, the treatment can result in a 5, 10, 15, 20, 25, 30, 35, 40, 45, 50, 55, 60, 65, 70, 75, 80, 85, 90, 95, 96, 97, 98, 99, or 100% reduction of the disease or symptoms relating thereto.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human

1. Compositions and Methods

Numerous cytokines have been implicated in pulmonary fibrosis. IL-13 has been shown to increase fibrotic activity in bleomycin-induced pulmonary fibrosis and radiation-induced lung fibrosis. Herein is shown that IL-17A-producing CD4+ T cells are significant in bleomycin-induced pulmonary fibrosis, and IL-17A is significantly increased in the bronchoalveolar lavage (BAL) of IPF patients. Also, CD4+PD-1+ T cells are increasingly present during sarcoidosis pulmonary progression, and that Th17 cells are the subjects with the greatest percentage of PD-1 expression.

In addition to IL-17A production, TGF-β has been shown to contribute to bleomycin-induced pulmonary fibrosis by inducing proliferation of fibroblasts and increasing collagen-production from myofibroblasts, as well as augmenting the development of Th17 cells. Microarray analyses demonstrating impaired transplant-free survival (TFS) among IPF patients with reduced gene expression in the co-stimulation and T cell activation pathway, such as CD28, suggests that PD-1 may have a role in pulmonary fibrosis. PD-1 suppresses T cell function primarily by inactivating CD28 signaling, in chronic conditions such as lung cancer. Prior to the present disclosure, a link establishing PD-1 and IL-17A interactions in pulmonary fibrosis had not been established. Accordingly, in one aspect, disclosed herein are methods of treating a pulmonary disease (for example, an interstitial lung disease such as idiopathic pulmonary fibrosis, bleomycin-induced pulmonary fibrosis, or sarcoidosis) in a subject comprising administering to the subject an agent that inhibits IL-17A and/or TGFβ-1.

As shown herein, co-culture of CD4+ T cells with human lung fibroblasts induces collagen-1 production; and blockade of the PD-1 pathway significantly decreases both TGF-β and IL-17A production, with concurrent reductions in collagen-1 production and thus treats pulmonary fibrosis. Thus, it is understood and herein contemplated that pulmonary fibrosis can be treated by blockade of the PD-1 pathway as this blockade reduces collagen-1 production. Accordingly, in one aspect, disclosed herein are methods of treating a pulmonary disease (for example, an interstitial lung disease such as idiopathic pulmonary fibrosis, bleomycin-induced pulmonary fibrosis, or sarcoidosis) in a subject comprising administering to the subject an agent that inhibits the interaction of PD-1 and PDL-1 (such as, for example, an anti-PD1 antibody, including, but not limited to nivolumab, pembrolizumab, pidilizumab, atezolizumab, avelumab, durvalumab, and BMS-936559).

Molecular analysis for significant pathways reveals that PD-1 regulates the critical transcription factor, STAT3. Blockade of the PD-1 pathway reduced STAT3 expression and chemical inhibition of STAT3 significantly inhibited collagen-1 production in co-culture experiments. Together, these studies demonstrate that despite distinct etiologies, common immunologic overlap of PD-1 regulation of IL-17A and TGF-β expression emerge among these three conditions of pulmonary fibrosis. Due to role of STAT3 in collagen-1 production in pulmonary fibrosis, it is understood and herein contemplated that one way to treat pulmonary fibrosis is through the reduction of STAT3 production. Thus, in one aspect, disclosed herein are methods of treating a pulmonary disease (for example, an interstitial lung disease such as idiopathic pulmonary fibrosis, bleomycin-induced pulmonary fibrosis, or sarcoidosis) in a subject comprising administering to the subject an agent that reduces STAT3 production or activation. Examples of STAT3 inhibitors are well known in the art. For example, STAT3 inhibitors can include Tyrosine phosphorylation inhibitors (Stattic, JSI-124, Withacnistin, and/or ibrutinib), SH2 domain inhibitors or dimerizaton inhibitors (PpYLKTK, SS 610, S31-201, S31-M2001, and/or STA-21), and/or DNA binding domain inhibitors (IS3 295, CPA-1, CPA-7, and/or Galielllalactone). Thus, in one aspect, disclosed herein are methods of treating a pulmonary disease (for example, an interstitial lung disease such as idiopathic pulmonary fibrosis, bleomycin-induced pulmonary fibrosis, or sarcoidosis) in a subject comprising administering to the subject an agent that reduces STAT3 production or activation; wherein the agent that reduces STAT3 production or activation is a STAT3 inhibitor selected from the group comprising Tyrosine phosphorylation inhibitors (Staitic, JSI-124, Withacnistin), SH2 domain inhibitors or dimerizaton inhibitors (PpYLKTK, SS 610, S31-201, S31-M2001, STA-21), and/or DNA binding domain inhibitors (IS3 295, CPA-1, CPA-7, Galielllalactone).

In one aspect, disclosed herein are methods and compositions of treating a pulmonary disease comprising administering to a subject with a pulmonary disease an agent, wherein the agent is a small molecule, peptide, polypeptide, siRNA, or antibody.

In one aspect, it is understood and herein contemplated that treatment of pulmonary fibrosis occurs due to a reduction in production of collagen-1. Accordingly, it is understood that any of the methods of treating pulmonary fibrosis disclosed herein similarly reduce production of collagen-1. Thus, in one aspect, disclosed herein are methods of reducing collagen-1 production in a subject with a pulmonary disease comprising administering to the subject an agent that reduces STAT3 production or activation, blocks the interaction of PD-1/PD-L1, and/or inhibitor IL-17A and/or TGFβ-1.

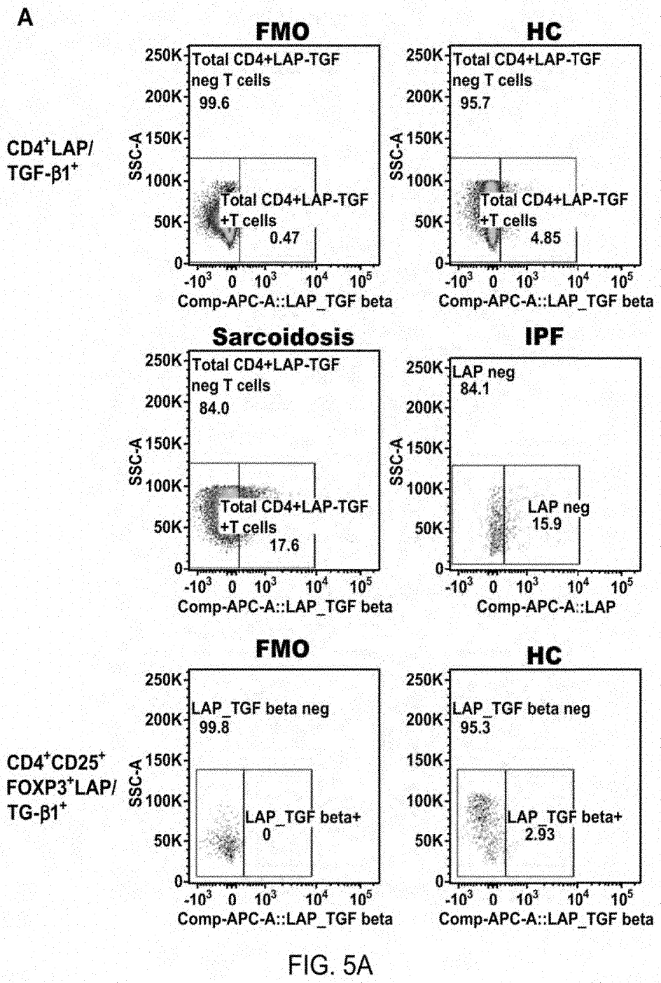

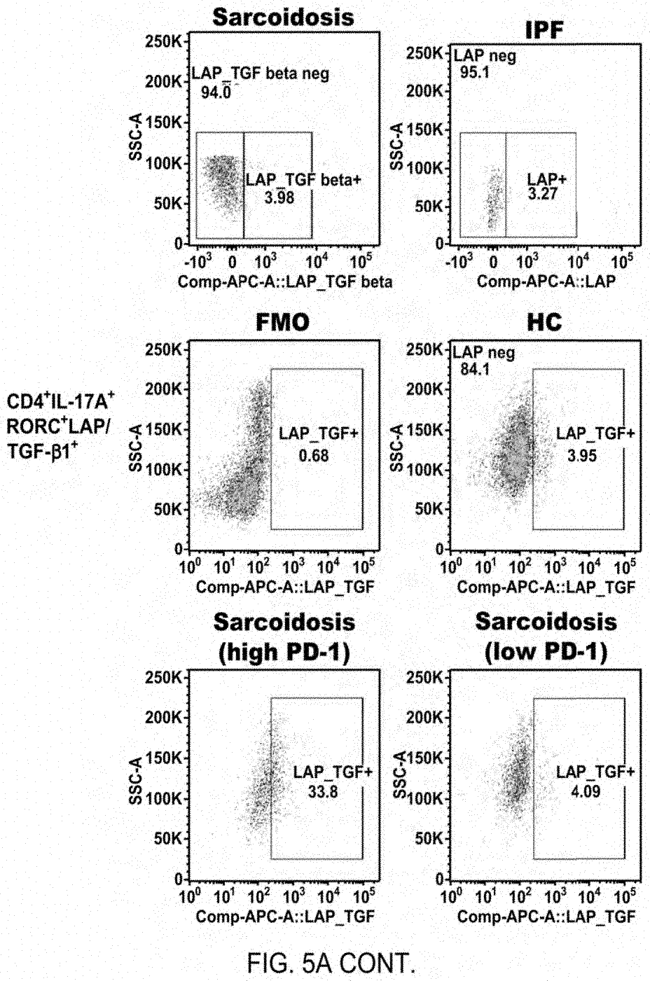

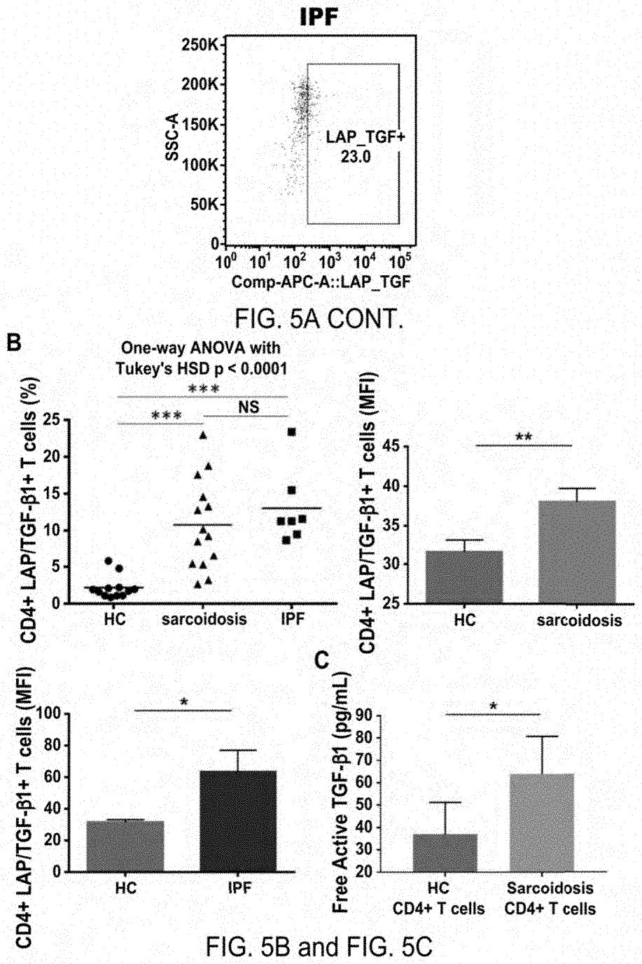

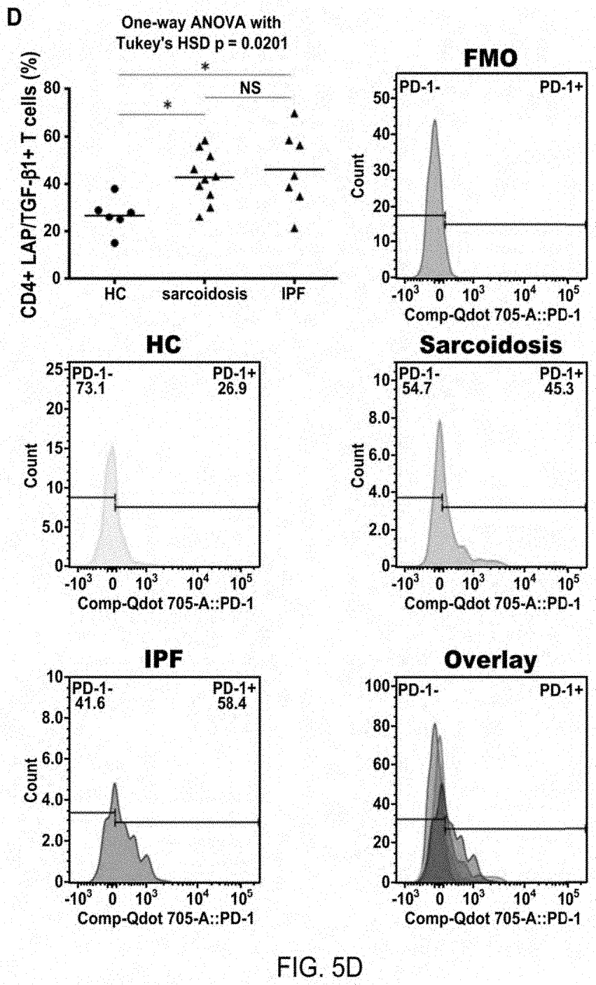

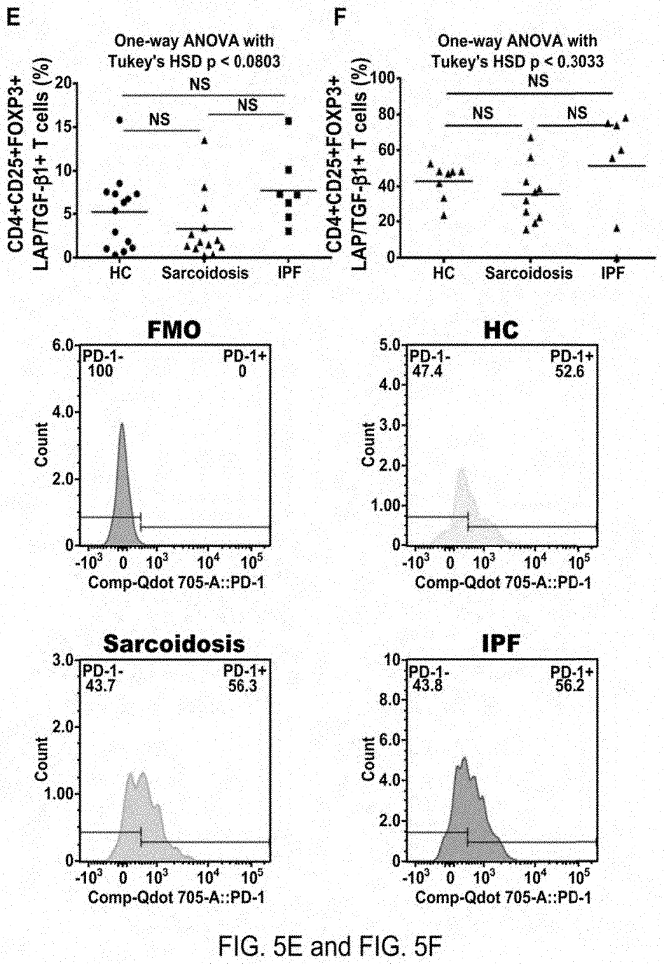

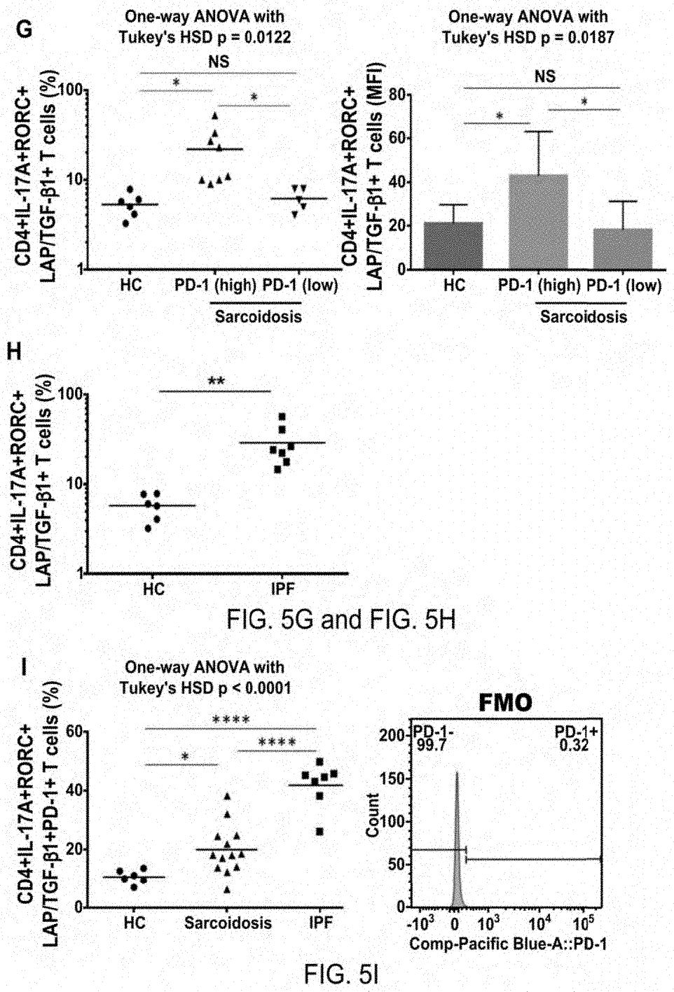

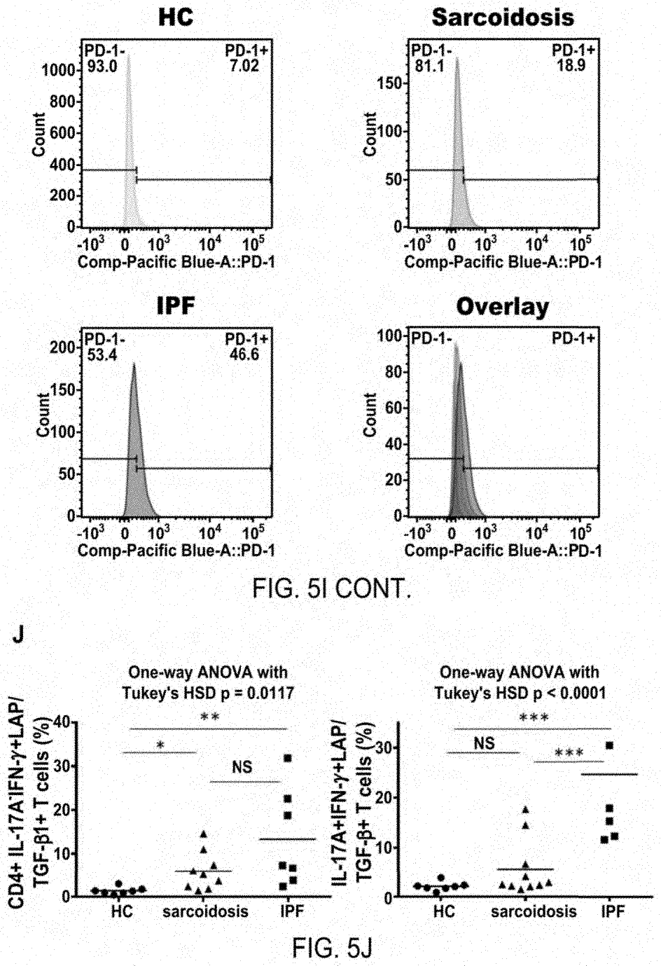

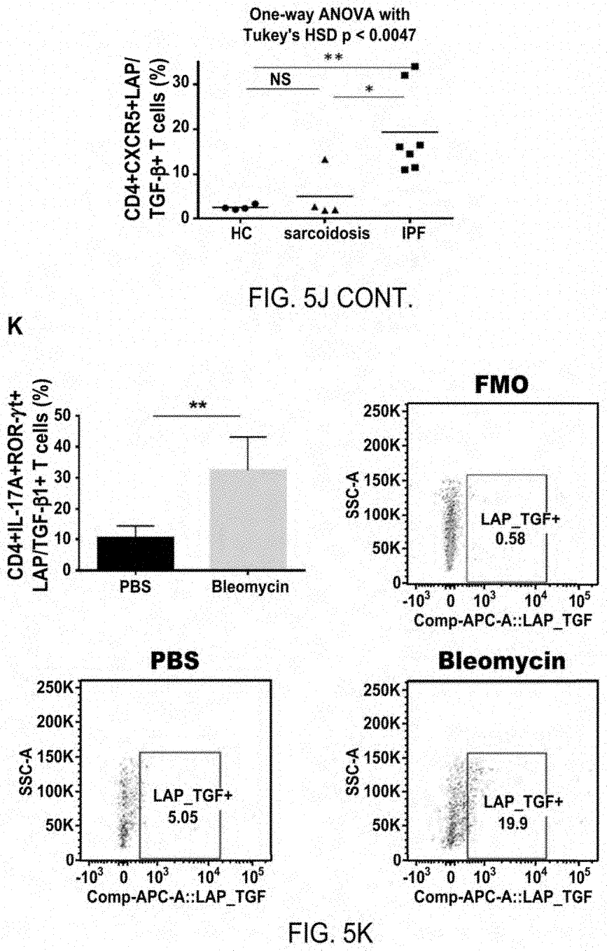

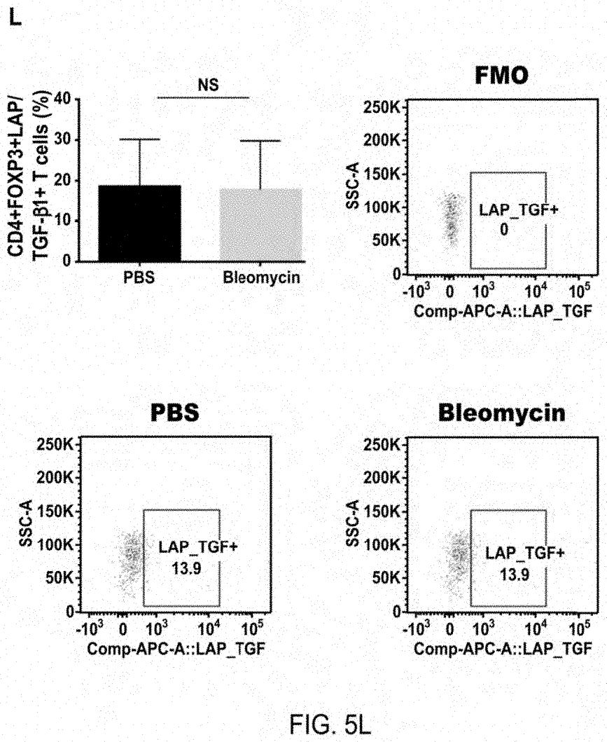

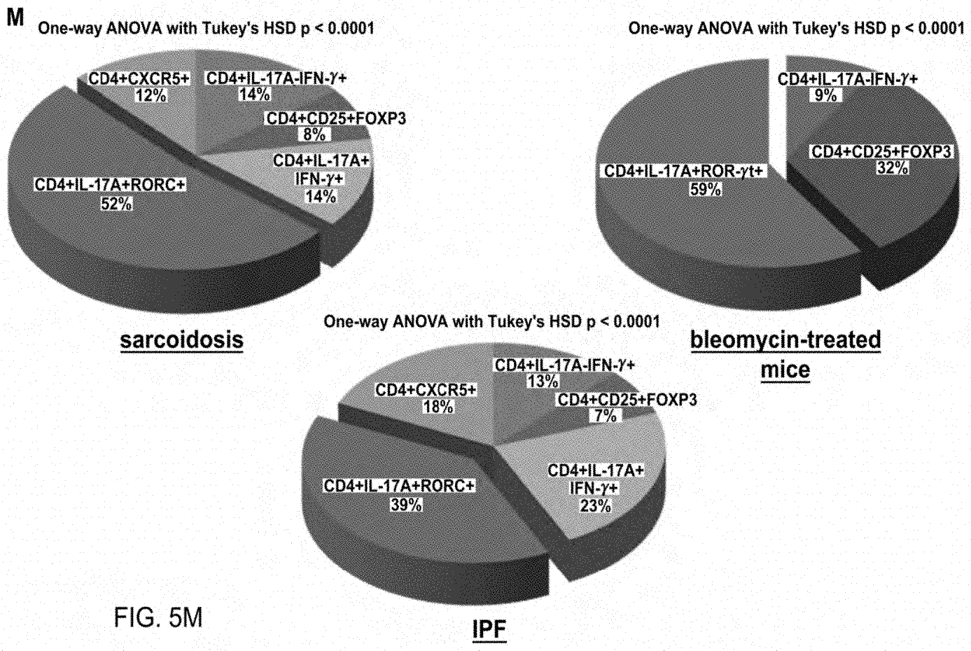

The knowledge of the effect of the immunological conditions associated with pulmonary fibrosis and the immunological factors that promote collagen-1 production provides a basis for assaying tissue from a subject with pulmonary fibrosis to assess the prognosis of the disease. Accordingly, in one aspect, disclosed herein are methods of creating a prognosis for a subject with a pulmonary disease comprising obtaining a tissue sample form a subject, isolating the TGFβ+PD1+CD4 T cells (T_{H}17 cells), measuring the number of T_{H}17 CD4+ T cells, wherein an increase in TGFβ+PD1+T_{H}17 cells relative to a control and/or a decrease in IFNγ+IL-17A+T_{H}17 cells relative to a control indicates decreased survival chances for the subject. It is understood and herein contemplated that in the disclosed prognostic methods, T_{H}17 numbers can be assessed by any means known in the art, including, but not limited to microarray, flow cytometry, or ELISA. Similarly, as noted herein in FIGS. 5, 6, 7, and, 8, IL-17 expression levels are associated with more aggressive pulmonary disease (such as, for example, an interstitial lung disease such as idiopathic pulmonary fibrosis, bleomycin-induced pulmonary fibrosis, or sarcoidosis) and poor prognosis for a subject with pulmonary disease. Thus, in one aspect, disclosed herein are methods of creating a prognosis for a subject with a pulmonary disease comprising obtaining a tissue sample form a subject, isolating the TGFβ+PD1+CD4 T cells (T_{H}17 cells), measuring the IL-17 expression levels relative to a control, wherein an increase in IL-17 expression levels relative to a control indicates a more aggressive disease and decreased survival chances for the subject.

Also disclosed herein are that certain polymorphisms are associated with a disease state. These polymorphism include single nucleotide polymorphisms (SNPs) in the STAT3 gene (such as, for example, rs11079042, rs17881621, and/or rs80350541), IL-17RA gene (such as, for example, rs41482444), IL-17D gene (such as, for example, rs182051094 and/or rs41480348), ERAP2 gene (such as, for example, rs61731306, rs873723, and/or rs1047591), PDCD1 gene (such as, for example, rs143359677), and/or TGF-β1 gene (such as, for example, rs4669). By detecting the presence of a polymorphism in one on these genes, the risk of persistence or disease progression can be determined. Thus, in one aspect, disclosed herein are methods of creating a prognosis for a subject with a pulmonary disease comprising obtaining a tissue sample form a subject, isolating the TGFβ+PD1+CD4 T cells (T_{H}17 cells), assaying for single nucleotide polymorphisms (SNPs) in the STAT3 gene (such as, for example, rs11079042, rs17881621, and/or rs80350541), IL-17RA gene (such as, for example, rs41482444), IL-17D gene (such as, for example, rs182051094 and/or rs41480348), ERAP2 gene (such as, for example, rs61731306, rs873723, and/or rs1047591), PDCD1 gene (such as, for example, rs143359677), and/or TGF-β1 gene (such as, for example, rs4669), wherein the presence of an assayed SNP in the STAT3 gene, IL-17RA gene, PDCD1 gene, or TGF-β1 gene indicate an increased risk of disease progression, the presence of a assayed SNP in the IL-17D gene indicates increased risk of persistence, and the presence of an assayed SNP in the ERAP2 gene indicates increased risk of disease progression and persistence. It is understood and herein contemplated that any means known in the art for detecting SNPs can be utilized to create a prognosis. Accordingly, in one aspect, disclosed herein are methods of creating a prognosis for a subject with a pulmonary disease comprising assaying for SNPs of any preceding aspect, wherein the SNP is identified by allele-specific PCR, sequencing, single base extension (SBE), oligonucleotide ligation assay (OLA), Heated oligonucleotide ligation assay (HOLA), mass spectrometry, single-strand conformation polymophism, and/or electrophoresis.

2. Antibodies 1

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with PD-1 or PD-L1 such that PD-1 is inhibited from interacting with PD-L1. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, sFv, and the like, including hybrid fragments. Thus, fragments of the antibodies that retain the ability to bind their specific antigens are provided. For example, fragments of antibodies which maintain PD-1 or PD-L1 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti-PD1 or anti-PD-L1 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

3. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

4. Immunoassays and Fluorochromes

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-1 methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor350™; Alexa Fluor430™; Alexa Fluor488™; Alexa Fluor 532™; Alexa Fluor546™; Alexa Fluor568™; Alexa Fluor594™; Alexa Fluor 633™; Alexa Fluor 647™; Alexa Fluor660™; Alexa Fluor680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, N G, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner Exemplary techniques are described in Ornstein L., Disc electrophoresis-I: Background and theory, Ann. NY Acad. Sci. 121:321-349 (1964), and Matsudiara, P T and D R Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelshfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immune complexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immune complex (antigen/antibody) formation. Detection of the immune complex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialised chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, Hayward, Calif.), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, Ariz.), rolling circle DNA amplification (Molecular Staging, New Haven Conn.), mass spectrometry (Intrinsic Bioprobes; Ciphergen, Fremont, Calif.), resonance light scattering (Genicon Sciences, San Diego, Calif.) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, Calif.; Clontech, Mountain View, Calif.; BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, Calif.; Biosite, San Diego, Calif.). In addition to the conventional antibodies, Fab and scFv fragments, single V-domains from camelids or engineered human equivalents (Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, Mass.) and ‘Anticalins’ based on the lipocalin structure (Pieris Proteolab, Freising-Weihenstephan, Germany) These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, Calif.).

Another methodology which can be used diagnostically and in expression profiling is the ProteinChip® array (Ciphergen, Fremont, Calif.), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulfide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### B. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: PD-1 Induces IL-17A and TGF-β Through STATS, Representing a Therapeutic Target Against Pulmonary Fibrosis

Herein identified are relevant immunologic mechanisms of pulmonary fibrosis in human and murine models, with a specific focus on PD-1. In order to achieve this, three distinct model systems were employed: the murine model of bleomycin-induced pulmonary fibrosis, sarcoidosis and idiopathic pulmonary fibrosis (IPF). PD-1+CD4+ T cells with reduced proliferative capacity are present in C57Bl6 mice following bleomycin administration, as well as in IPF patients and sarcoidosis patients experiencing disease progression Immunohistochemical analysis for PD-1 upregulation is noted on IPF lymphocytes and epithelial cells. These PD-1+CD4+ T cells produce both TGF-β and IL-17A. Assessment for TGF-β expression reveals that Th17 cells are the greatest TGF-β producers among the CD4+ T cell subsets in all three groups. Co-culture of CD4+ T cells with human lung fibroblasts induces collagen-1 production; blockade of the PD-1 pathway significantly decreases both TGF-β and IL-17A production, with concurrent reductions in collagen-1 production. Molecular analysis for significant pathways reveals that PD-1 regulates the critical transcription factor, STAT3. Blockade of the PD-1 pathway reduced STAT3 expression and chemical inhibition of STAT3 significantly inhibited collagen-1 production in co-culture experiments. Together, these studies demonstrate that despite distinct etiologies, common immunologic overlap of PD-1 regulation of IL-17A and TGF-β expression emerge among these three conditions of pulmonary fibrosis. Moreover, these findings establish the biological significance of the PD-1 pathway, supporting targeting of either STAT3, IL-17A or the PD-1 pathway as a potential therapeutic treatment for patients suffering from pulmonary fibrosis.

a) Results

(1) Microarray Analysis Demonstrates Overexpression Genes within IL-17 Signaling Pathway Associated with Poor Outcome in IPF Patients

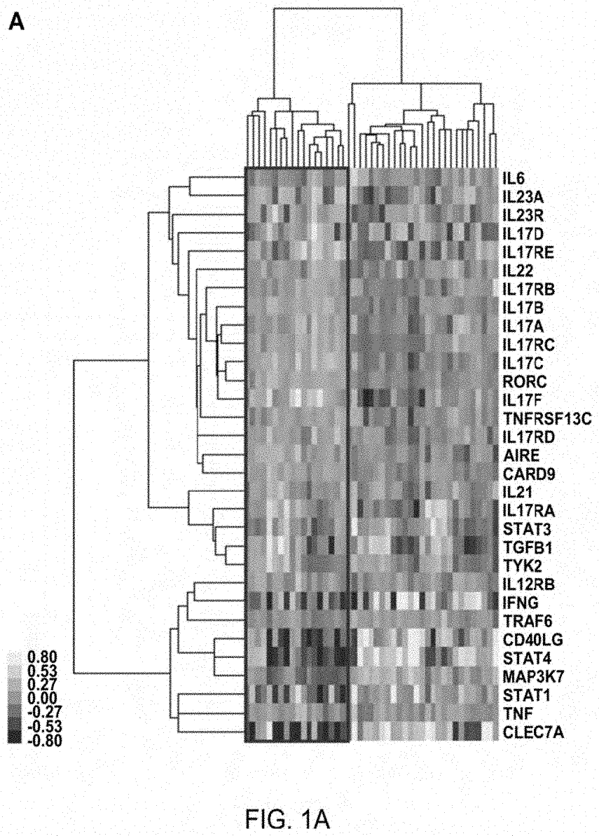

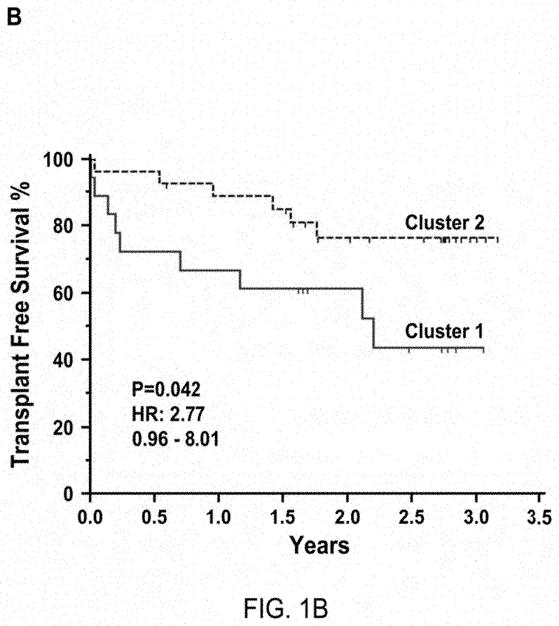

Because increased IL-17A has been noted in the bronchoalveolar lavage of IPF patients, the molecular evidence of increased IL23/IL-17 signaling with IPF specimens was assessed. A microarray gene expression dataset was downloaded from the National Center for Biotechnology Information's Gene Expression Omnibus (GEO). In this study, total RNA was extracted from PBMC and hybridized to Affymetrix GeneChip microarrays in 47 IPF patients. Peripheral blood mononuclear cell (PBMC) gene expression profiles predictive of poor outcomes in IPF were identified by performing microarray experiments of PBMCs in discovery and replication cohorts of IPF patients. Microarray analyses identified 52 genes associated with transplant-free survival (TFS) in the discovery cohort. Clustering the microarray samples of the replication cohort using the 52-gene outcome-predictive signature distinguished two patient groups with significant differences in TFS (FIG. 1A). The overexpression of critical mediators of IL-17A expression is associated were identified with ˜30% reduction in TFS (FIG. 1B). These findings support further evaluation of the role of the IL-17A signaling pathway in IPF pathogenesis.

(2) Genetic Polymorphisms in the STAT3, IL17RA and TGF-β1 Loci Correlate with Risk of Developing Interstitial Lung Disease

Th17 cells are essential for host defense at mucosal surfaces, such as the gut and lungs. 685,252 SNPs residing within 43 gene regions that have been identified as being involved with the Th17 pathway were analyzed in a data set comprised of 2,918 sarcoidosis. Association with case/control status was tested, as well as used a subset of the subjects to test for association with persistence of disease (623 persistent cases, 308 resolved cases). All analysis was performed using EMMAX. Three gene regions were found to contain SNPs with a suggestive association to case/control status, and two gene regions contained SNPs with a suggestive association to persistence of disease. Sarcoidosis patients with SNPs (rs11079042) in the STAT3 gene, (rs41482444) IL17RA and (rs4669) TGF-β1 gene had significantly higher risk for sarcoidosis disease progression (p=6.66E-05, p=4.09E-04, and 1.96E-04; respectively), implying Th17 pathway involvement in sarcoidosis disease advancement (Table 1). Of these genes only ERAP2 is suggestive under both phenotypes, while IL-17D increases in significance when narrowing from cases/control to persistence phenotype; becoming suggestive (Table 2). STAT3, however, is suggestive in disease case/control but not in persistence. This indicates a substantial difference in mechanism between development of disease and resolution of disease. Notably, transcription factors for STAT3 are greatly enriched in 17 of ˜40 genes included in the targeted sequencing, even after adjusting for multiple testing (q=0.022).

TABLE 1
Single nucleotide polymorphisms (SNPs) in
Th17-associated genes in sarcoidosis.

| GENE | CHR | Position | SNP | P-value | Source |
|---|---|---|---|---|---|
| PRDM1 | 6 | 106855806 | rs6568431 | 7.42E−06 | GWAS |
| STAT3 | 17 | 40467179 | rs11079042 | 6.66E−05 | GWAS |
| CSF2RB2 | 22 | 37372327 | rs6519043 | 4.02E−05 | GWAS |
| ERAP2 | 5 | 96239234 | rs61731306 | 4.37E−05 | Exome |
| IL17RA | 22 | 17590404 | rs41482444 | 4.09E−04 | Exome |
| TGFB1 | 5 | 133392426 | rs4669 | 1.96E−04 | Exome |

TABLE 2
Single nucleotide polymorphisms (SNPs) associated
with sarcoidosis progression.

| chr | gene | cc snp | cc p-value | rvp snp | rvp p-value |
|---|---|---|---|---|---|
| 17 | STAT3 | rs17881621 | 3.15E−06 | rs80350541 | 7.58E−013 |
| 5 | ERAP2 | rs873723 | 9.78E−07 | rs10475791 | 4.29E−07 |
| 6 | TNF | rs56826533 | 2.09E−05 | rs56826533 | 3.18E−03 |
| 13 | IL-17D | rs182051094 | 2.53E−04 | rs41470348 | 6.52E−05 |

(3) PD-1+CD4+ T Cells with Diminished Proliferative Capacity are Present in Interstitial Lung Disease Subjects, as Well as Mice with Pulmonary Fibrosis

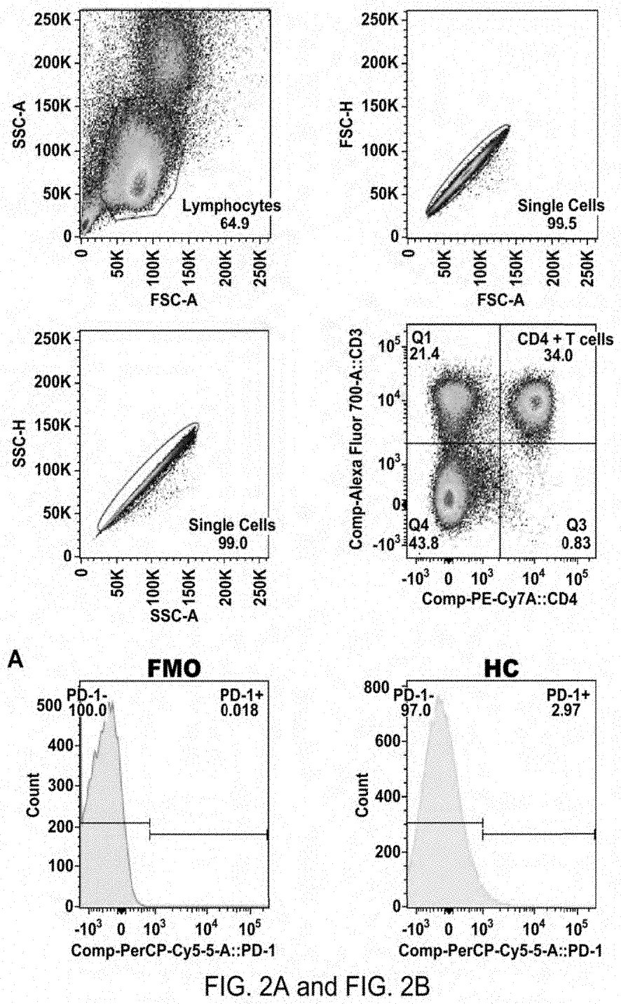

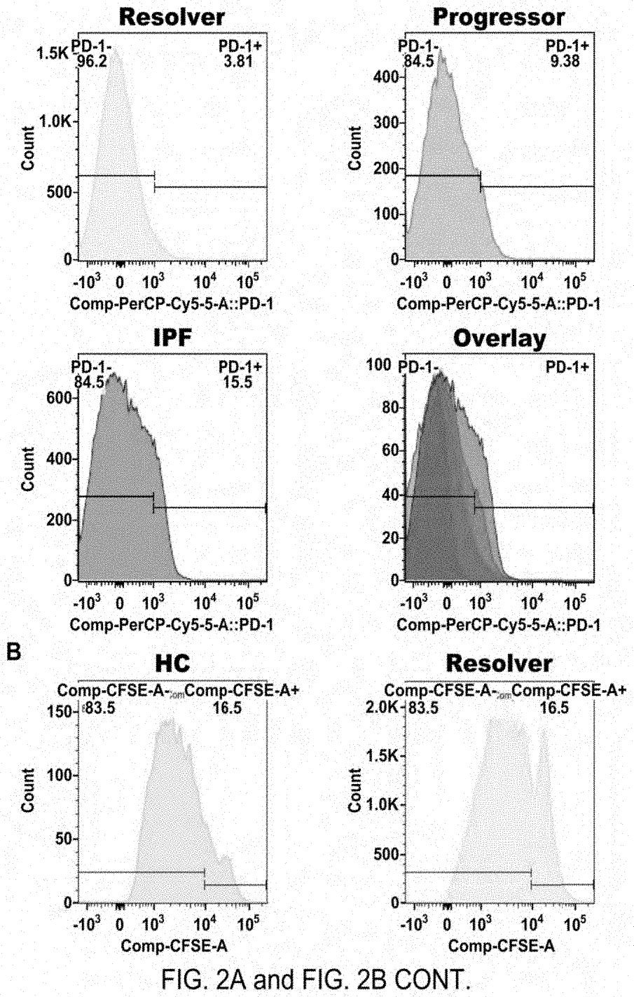

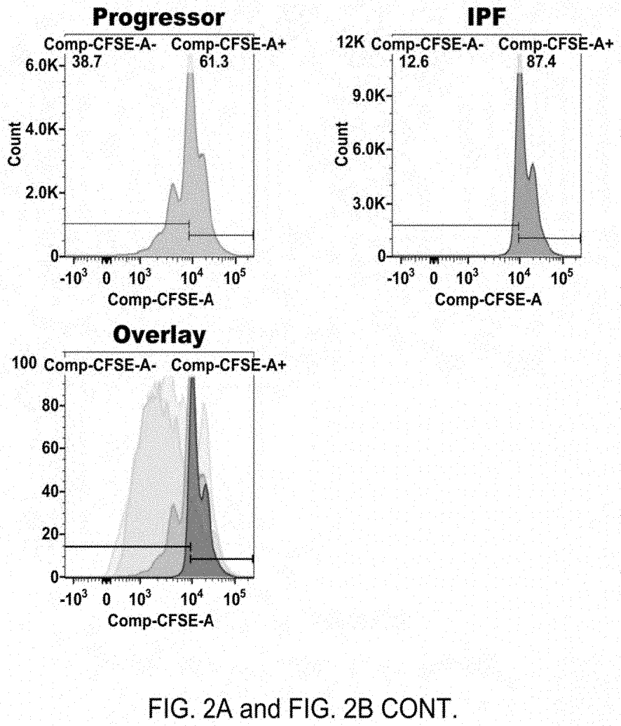

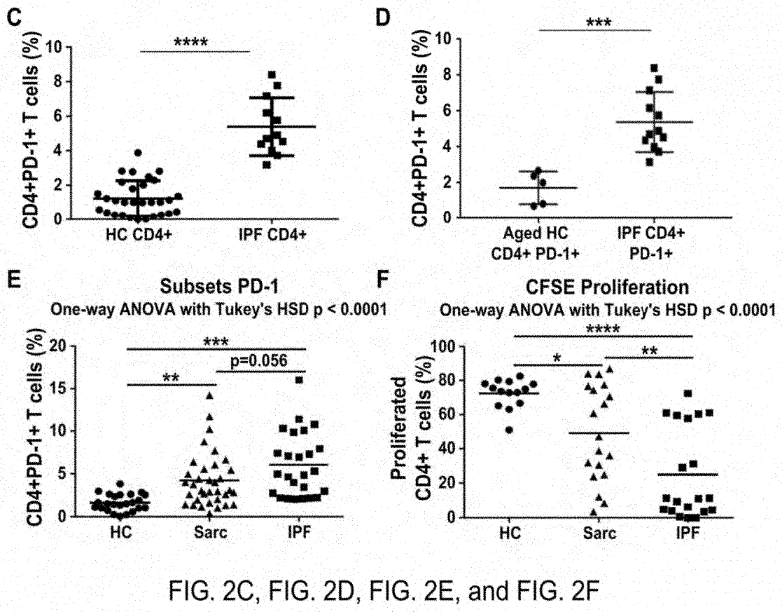

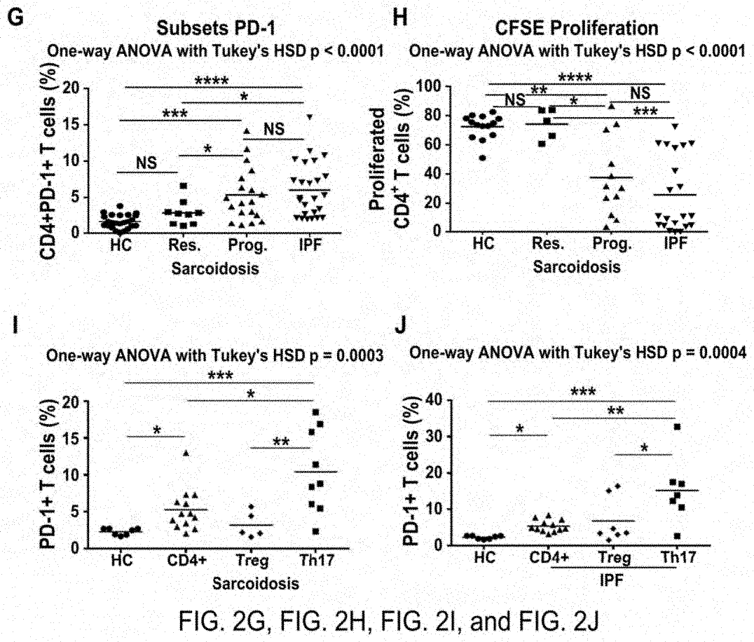

While it is unlikely that the adaptive immune system is the lone contributor in pulmonary fibrosis, there is a role for CD4+ T cells. The presence of autoreactive CD4+ T cells have been demonstrated in patients with interstitial lung disease, including IPF and sarcoidosis. CD28-deficiency in mice has been reported to impair the development of fibrosis after bleomycin administration. Furthermore, adoptive transfer of CD28-positive T cells into CD28-deficient mice recuperates fibrotic development succeeding bleomycin treatment. Molecular evidence that the balance between costimulatory and co-inhibitory signals shaping T cell exhaustion coincide with clinical outcomes in idiopathic diseases, such as IPF. Progressive increases in PD-1+CD4+ T cells of sarcoidosis subjects experiencing disease progression has also been noted. To further investigate co-inhibitors in interstitial lung disease, three models of pulmonary fibrosis were examined for PD-1 expression: IPF, sarcoidosis and bleomycin-induced pulmonary fibrosis. PD-1 upregulation is also significantly higher on IPF CD4+ T cells, compared to healthy controls (HC) (p<0.0001) (FIG. 2A-C). Increased PD-1 expression on IPF CD4+ T cells was still apparent after adjusting for age disparities between HC and IPF patients (p<0.0001) (FIG. 2D). PD-1 upregulation was accompanied by reduced proliferative capacity amongst the disease cohorts when compared to HC CD4+ T cells (p<0.0001) (FIG. 2E-F). More importantly, PD-1 upregulation on sarcoidosis and IPF CD4+ T cells correlated with disease progression and loss of immune function (p<0.0001) (FIG. 2G-H) and by significant reductions in proliferative capacity in IPF PD-1+CD4+ T cells (p<0.0001, unpaired two-tailed t-test). The CD4+ T cell subsets were defined demonstrating the highest percentages of PD-1+ T cells. Intriguingly, Th17 cells expressed the highest percentage of PD-1+ cells in both sarcoidosis and IPF patients (p=0.0003, p=0.0004; respectively (FIG. 2I-J).

(4) Genetic Polymorphisms in the PDCD1 Loci Correlate with Risk of Developing Interstitial Lung Disease

Nivolumab is a programmed death 1 (PD-1) inhibitor. PD-1/PD-L1 inhibitors are antibodies that block immune checkpoint molecular (i.e., PD-1 and PD-L1) and serve to restore T-cell immune responses against tumors. Association with case/control status was tested, as well as used a subset of the subjects to test for association with persistence of disease. All analysis was performed using EMMAX. The PCDC1 gene region was found to contain SNPs with a suggestive association to case/control status and progression of disease. Sarcoidosis patients, post-inflammatory pulmonary fibrosis patients, idiopathic fibrosis alveolitis patients, and other alveolar and parietoalveolar pneumonopathy patients with SNPs (rs1143359677) in the the PDCD1 gene had significantly higher risk of disease progression (see odds ration in Table 3), implying PD1/PDL1 checkpoint pathway involvement in pulmonary disease advancement (Table 3). The data in Table 3 indicate that the SNP is functioning like the opposite of the drug. So anything with a positive Odds Ratio (OR) is a new indication and checkpoint inhibitor blockade is a viable therapeutic target to optimize clinical outcomes and thus can be used treat the indication.

TABLE 3

| rsID | Gene | Phecode | Phenotyppe | cases | controls | odds_ratio | P | AFF_11 | AFF_12 |
|---|---|---|---|---|---|---|---|---|---|
| rs143359677 | PDCD1 | 697 | Sarcoidosis* | 295 | 24926 | 8.4890 | 0.0004683 | 0 | 5 |
| rs143359677 | PDCD1 | 502 | Postinflammatory | 396 | 19673 | 4.9760 | 0.01102 | 0 | 4 |
| | | | pulmonary | | | | | | |
| | | | fibrosis | | | | | | |
| rs143359677 | PDCD1 | 504 | Other alveolar | 214 | 19673 | 6.9190 | 0.01118 | 0 | 3 |
| | | | and | | | | | | |
| | | | parietoalveolar | | | | | | |
| | | | pneumonopathy | | | | | | |
| rs143359677 | PDCD1 | 504.1 | Idiopathic | 142 | 19673 | 6.9520 | 0.03667 | 0 | 2 |
| | | | fibrosing | | | | | | |
| | | | alveolitis | | | | | | |

Key:
AFF_11 homozygotes;
AFF_12 heterozygotes

(5) Bleomycin Murine Model Reveals Reduced Fibrosis in PD-1 Null Mice

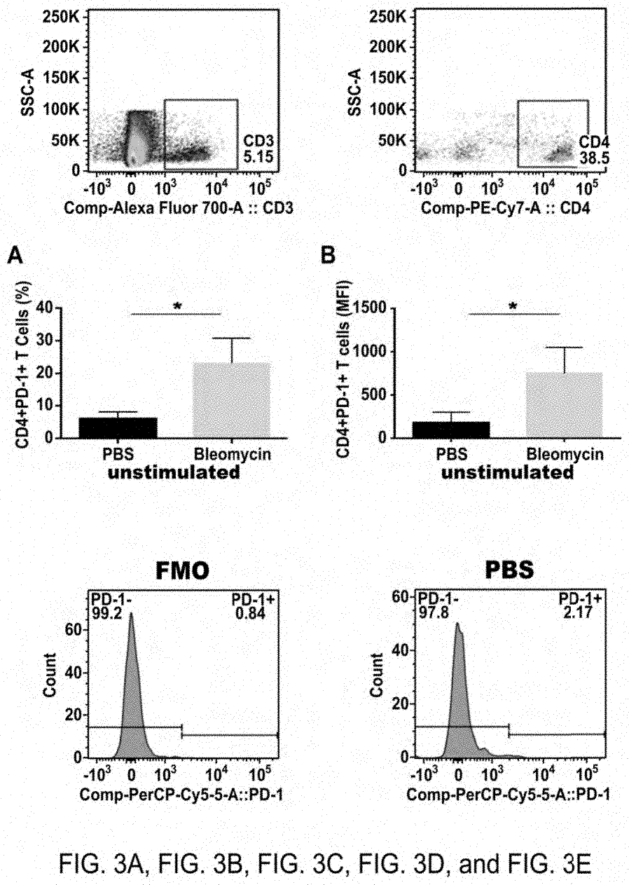

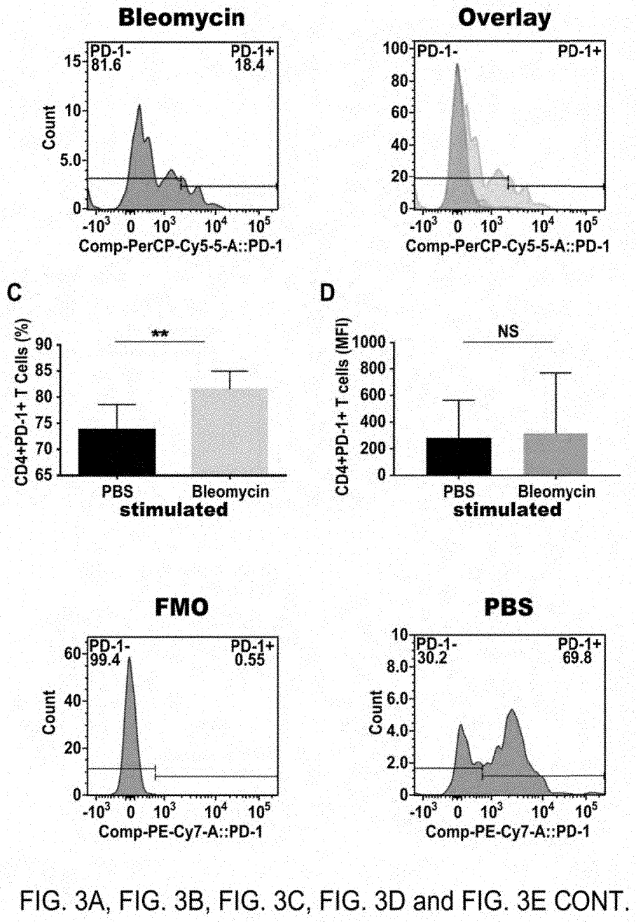

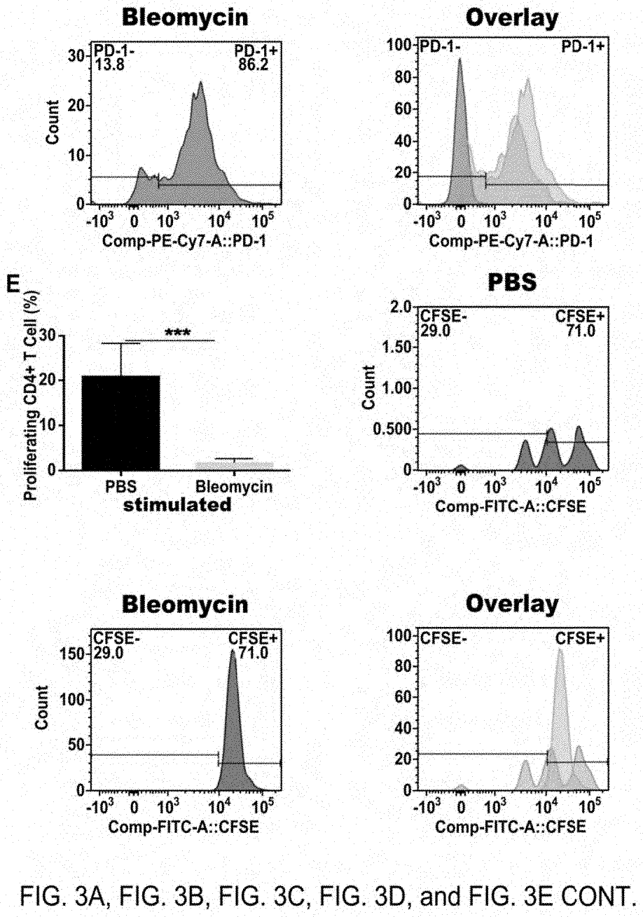

PD-1 upregulation and diminished effector function was also noted on CD4+ T cells from single cell lung suspensions of bleomycin-treated C57BL/6J mice (FIG. 3). The percentage of unstimulated CD4+ T cells expressing PD-1 and mean fluorescent intensity (MFI) was significantly higher at Day 21 in the bleomycin treated mice than the PBS-treated group (p=0.02) (FIG. 3A-B). Following T cell receptor (TCR) stimulation, both groups demonstrated increased PD-1 upregulation; however, a significantly higher percentage of CD4+ T cells expressing PD-1 persisted in the bleomycin-treated group (p=0.01) (FIG. 3C-D). Notably, PD-1 upregulation was accompanied by significantly diminished proliferative capacity in the bleomycin-treated CD4+ T cells, compared to those obtained from mice treated only with PBS (p=0.0009) (FIG. 3E). This observation establishes the functional significance of PD-1 upregulation on CD4+ T cells following bleomycin administration.

Th17 cells from single cell lung suspensions of bleomycin-treated mice also displayed increased LAP/TGF-β1 secretion in comparison to the control mice (p<0.01). Furthermore, no significant differences in TGF-β1 expression were apparent between Tregs from the control and bleomycin-treated mice (p=0.92). When comparing TGF-β1 secretion from CD4+ T cells following bleomycin administration, it was observed that Th17 cells were the main producers of LAP/TGF-β amongst the CD4+ T cell subsets. The detection of PD-1+CD4+ T cells in models of pulmonary fibrosis due to acute lung injury indicates that PD-1 pathway may be a common point of immunologic convergence in pulmonary fibrosis despite the etiology.

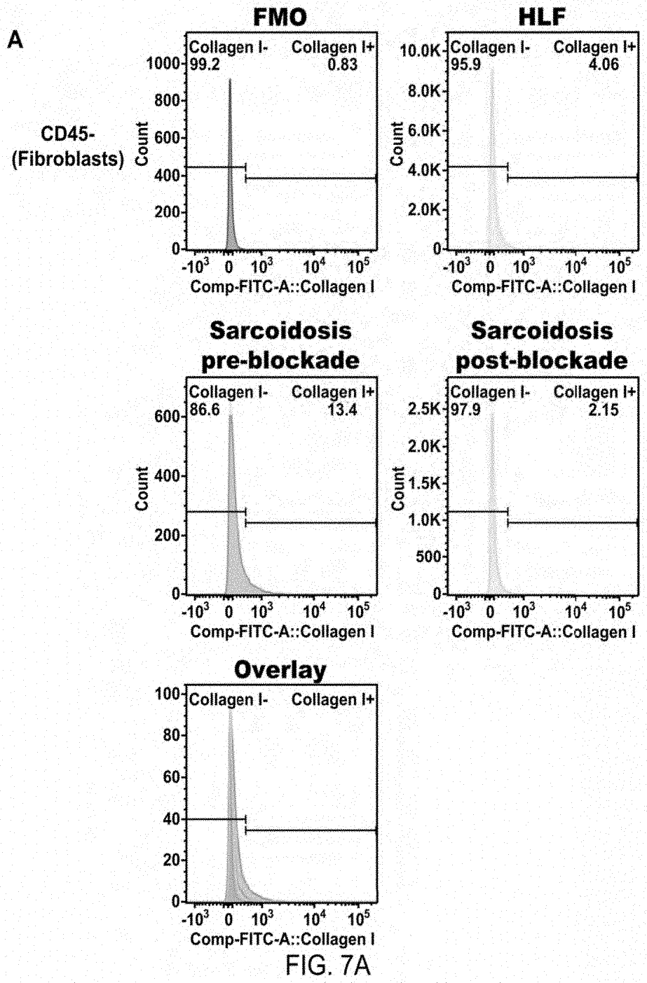

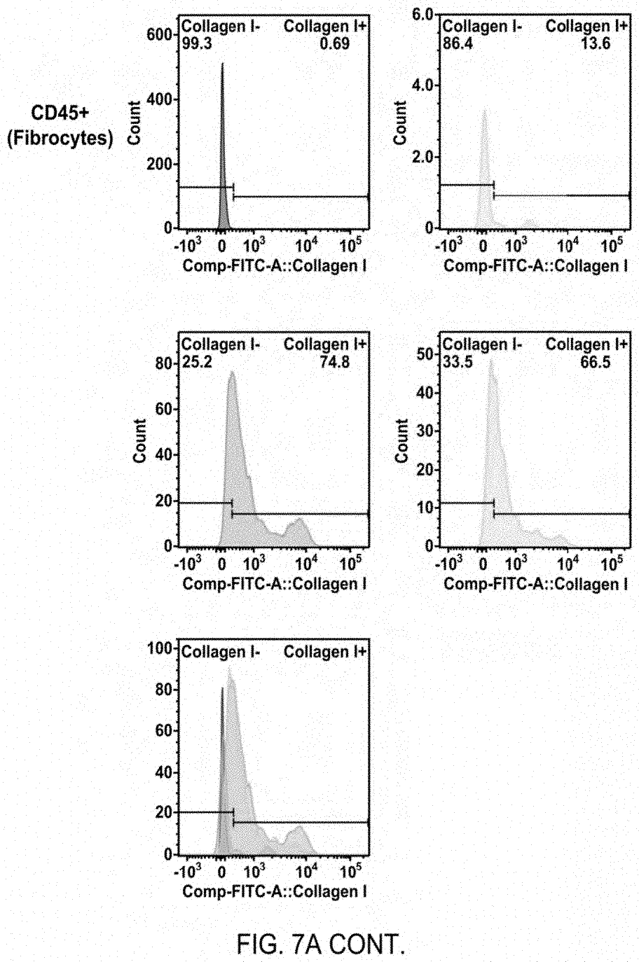

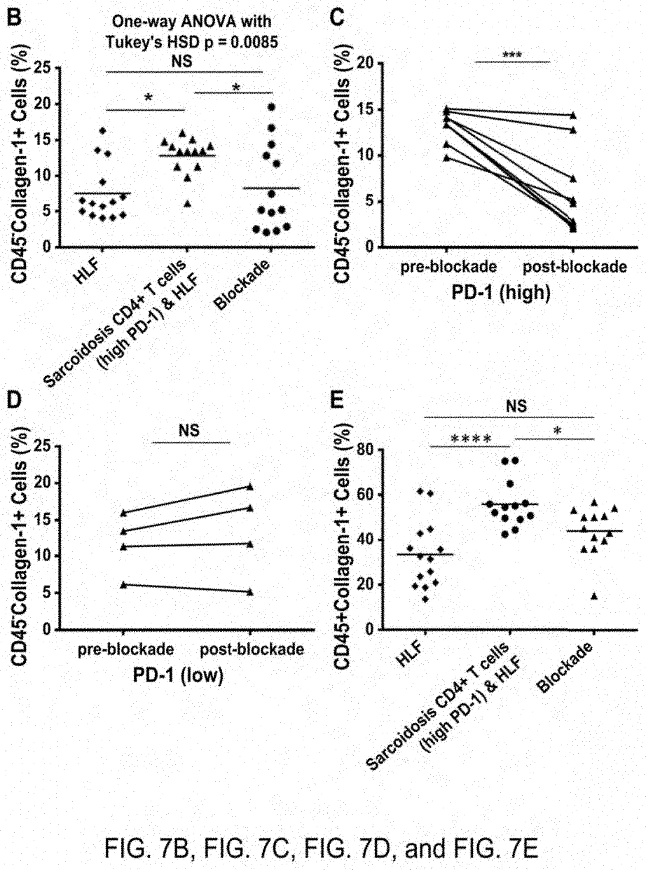

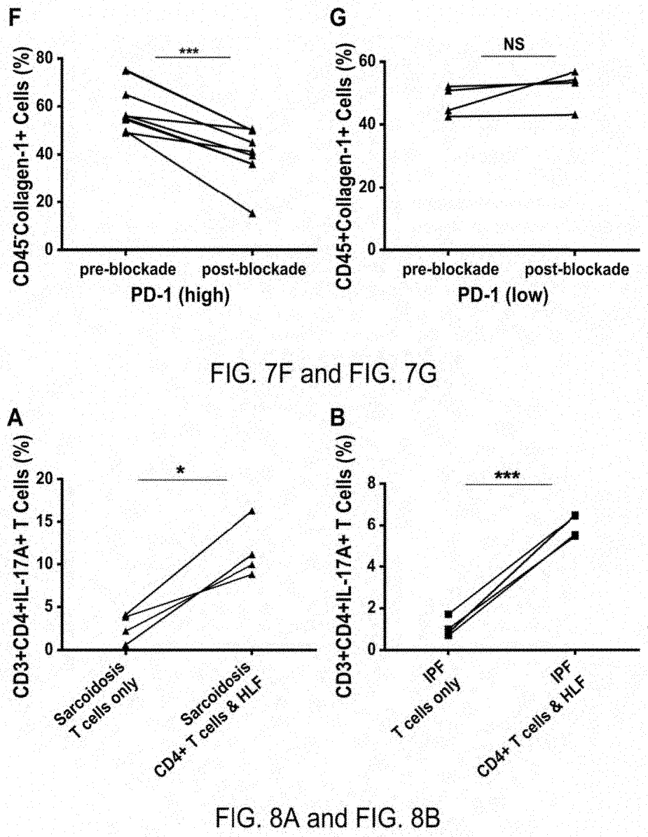

To extend the aforementioned human observations in vivo, wild-type and PD-1 null C57BL/6 mice were challenged with 0.2 mg of bleomycin via intranasal administration, then euthanized at day 14. To analyze fibrosis parameters, pulmonary trichrome staining and hydroxyproline content by HPLC were measured. Representative H&E and trichrome sections are shown in FIG. 7G. Fibrosis degree and severity were significantly higher by Ashcroft scoring in the WT C57BL6, compared to the PD-1 KO mice (P=0.002, unpaired two-tailed t-test) (FIG. 7H). Assessment of hydroxyproline content by HPLC confirmed the distinctions determined by the histologic scoring. Significantly lower levels of hydroxyproline were noted in PD-1 KO mice, compared to wild-type C57BL6 (41.7+17.0 versus 111.3+8.7, p=0.02, unpaired two-tailed t-test) (FIG. 7I). These findings provide in vivo confirmation of the relevance of the PD-1 pathway in interstitial lung disease.

(6) IFP Pulmonary Biopsies Demonstrate Increased PD-1 Expression in Lymphocytes and Epithelial Cells

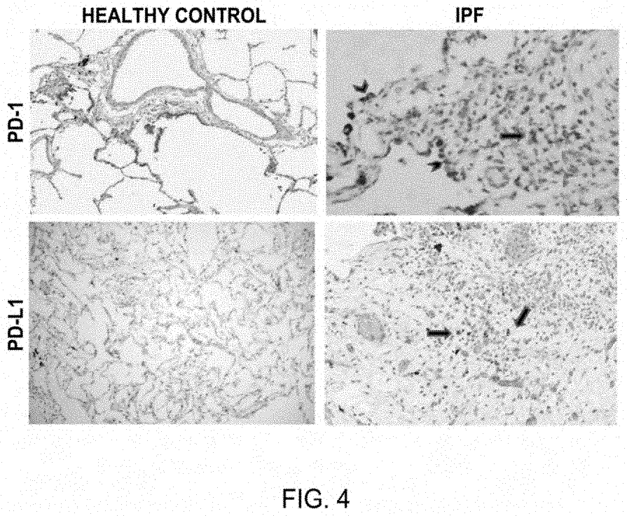

Sarcoidosis lung biopsies demonstrate increased PD-1 expression on lymphocytes and PD-L1 in granulomatous regions Immunohistochemical assessment for PD-1 expression for IPF human lung biopsies revealed increased PD-1 on lymphocytes, compared to healthy control lungs (FIG. 4—arrows). Even more compelling was the observation that PD-1 expression was present on lymphocytes in proximity to fibroblastic foci, but the strongest staining was noted on epithelial cells (FIG. 4—arrowheads). PD-L1 expression was also noted within the IPF biopsies (FIG. 4). Grading of severity of PD-1 expression on IPF and HC samples revealed that IPF specimens possessed significantly higher PD-1+ lymphocytes, than HC specimens (p=0.002, unpaired two-tailed t-test).

(7) Th17 Cells Display the Highest Expression of TGF-β1 Mediated by PD-1 Upregulation

TGF-β are multifunctional cytokines produced by many cell types, including lymphocytes. They are the primary factors involved in wound healing and tissue repair. TGF-β release induces the production of extracellular matrix (ECM) proteins and their stabilization. Under normal conditions, this ultimately results in tissue repair and restoration. Conversely, excessive TGF-β production in a dysregulated or diseased state results in a pathogenic phenotype that leads to organ fibrosis, and eventually, compromised organ function. TGF-β is synthesized and processed within the cell and then secreted in the form of the large latent TGF-β complex in which it, along with other proteins, is bound to latency-associated peptide (LAP). The presence of PD-1+CD4+ T cells in bleomycin-treated mice, IPF and sarcoidosis subjects led to an investigation of whether CD4+ T cells from these subjects secrete TGF-β, and if distinctions in TGF-β expression by subsets are present. Active TGF-β1 expression was ˜70% in sarcoidosis and IPF CD4+ T cell cultures compared to ˜30% in healthy controls (p=0.0097, one way ANOVA with Tukey's HSD). No significant distinction was observed in active TGF-β1 between the sarcoidosis and IPF CD4+ T cells. There was a greater percentage of total CD4+ T cells expressing membrane bound TGF-β1 (LAP/TGF-β1) in sarcoidosis and IPF patients, compared to HC (p<0.0001). LAP/TGF-β1 MFI was also significantly increased in sarcoidosis and IPF CD4+ T cells compared to HC (p<0.009, p=0.02; respectively) (FIG. 5A-B). Investigation of TGF-β1 in its active form revealed a significant increase in sarcoidosis CD4+ T cells, compared to healthy controls (FIG. 5C). This increase in LAP/TGF-β1 expression remained with assessing for PD-1 upregulation on these cells as well (p=0.02) (FIG. 5D). Thus, these findings demonstrate the presence of TGF-β-secreting CD4+ T cells with PD-1 upregulation in patients with fibrotic lung disease.

TGF-β promotes the development of Tregulatory cells through the essential transcription factor, FOXP3. T regulatory cells (T regs) are known to facilitate the development of fibrosis and possess immunosuppressive actions via the release of TGF-β1. In silica-induced pulmonary fibrosis, T regs accumulated in the lungs, expressed increased levels of TGF-β1, enhanced collagen accumulation and directly stimulated fibroblast proliferation. However, T reg neutralization in this animal model increased CD4+ T cell effectors and did not eradicate the aforementioned fibrotic features. Hence, it was investigated if increased TGF-β1 expression observed on CD4+ T cells in the disease models was the result of T regulatory cell activity. First, the overall percentages of T regs in the disease models was investigated. Significantly increased sarcoidosis T regs was present in the sarcoidosis but no significant differences between HC and IPF T regs (HC:sarc, p=0.001; HC: IPF, p=0.08; respectively). There were no significant differences in LAP/TGF-β1 being produced by sarcoidosis or IPF T regulatory cells compared to HC (p=0.08) (FIG. 5E). Interestingly, unlike in total CD4+ T cells (FIG. 5D), there were no distinctions in PD-1 expression on sarcoidosis or IPF T regs producing TGF-β1 (p=0.30) (FIG. 5F).

The pro-inflammatory cytokine IL-6 modulates TGF-β promotion of iTreg development in favor of Th17 development. LAP/TGF-β1 secretion from Th17 cells was therefore examined A significant difference in secreted LAP/TGF-β1 by sarcoidosis and IPF Th17 cells was noted, compared to HC (p=0.01) (FIG. 5G). Interestingly, the sarcoidosis Th17 cells that demonstrated increased expression of LAP/TGF-β1 were derived from patients with high baseline PD-1 MFI but not those with baseline PD-1 MFI consistent with HC (FIG. 5G). IPF Th17 cells also revealed increased LAP/TGF-β1 expression (p=0.003) (FIG. 5H). PD-1 was also significantly increased on Th17 cells that were TGF-β1+ from both sarcoidosis and IPF subjects compared to HC (p<0.0001) (FIG. 5I). Th1 cells that do not produce IL-17A had significantly higher LAP/TGF-β1 expression in sarcoidosis and IPF cells, compared to HC (p<0.01) (FIG. 5J). CD4+ T cells that were dual producers of IL-17A and IFNγ possessed significantly higher LAP/TGF-β1 expression in IPF cells, compared to HC, but the sarcoidosis subjects were not distinct (p<0.0001). T follicular helper cells producing LAP/TGF-β1 were higher in IPF cells, compared to HC, but the sarcoidosis subjects were not distinct (p<0.005) (FIG. 5J). Th17 cells from single cell lung suspension of bleomycin-treated mice also displayed increased LAP/TGF-β1 secretion in comparison to the control mice (p=0.003) (FIG. 5K). Furthermore, no significant differences in TGF-β1 expression was apparent between T regs from the control and bleomycin-treated mice (p=0.92) (FIG. 5L). Intracellular TGF-β1 levels were examined in both sarcoidosis and IPF models and noticed a significant difference in its expression in CD4+IL-17A+ T cells, compared to healthy controls that correlated with PD-1 upregulation. When comparing TGF-β1 secretion in the three disease models, it was observed that Th17 cells were the main producers of LAP/TGF-β amongst the CD4+ T cell subsets (FIG. 5M).

(8) PD-1 Pathway Blockade Reverses the Capacity of Sarcoidosis and IPF CD4+ T Cells to Induce Human Lung Fibroblast-Derived Collage-1 Production

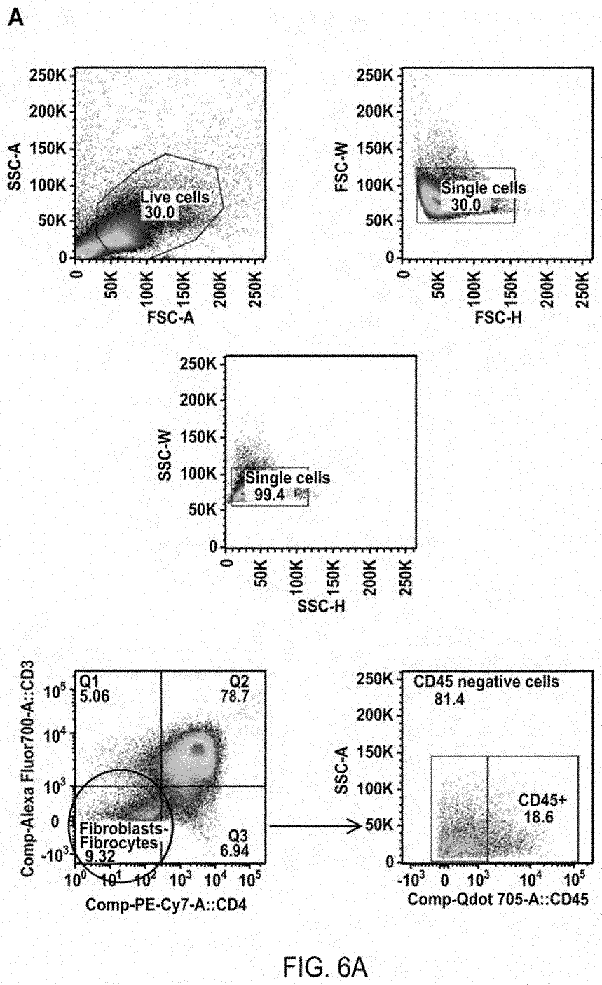

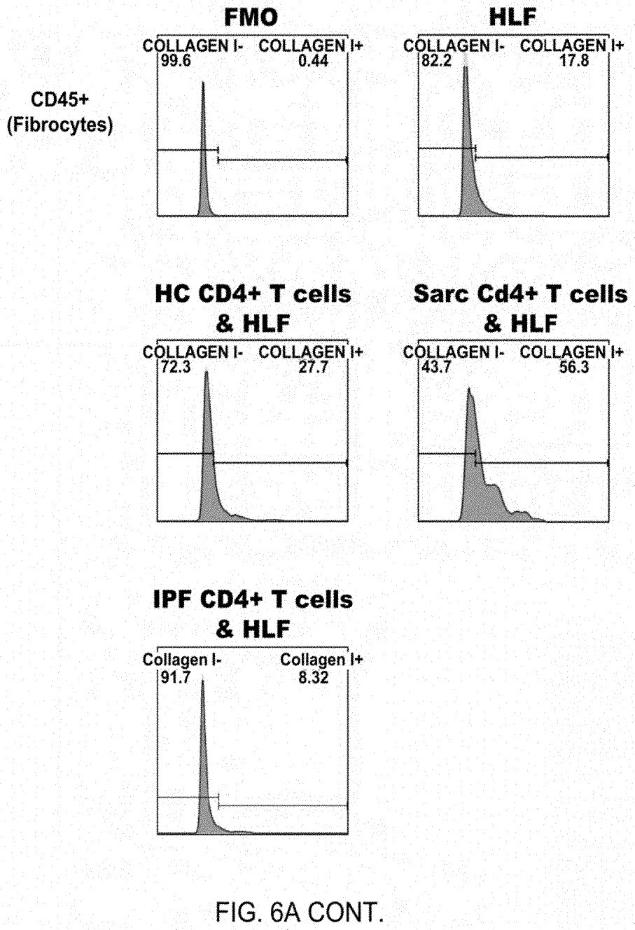

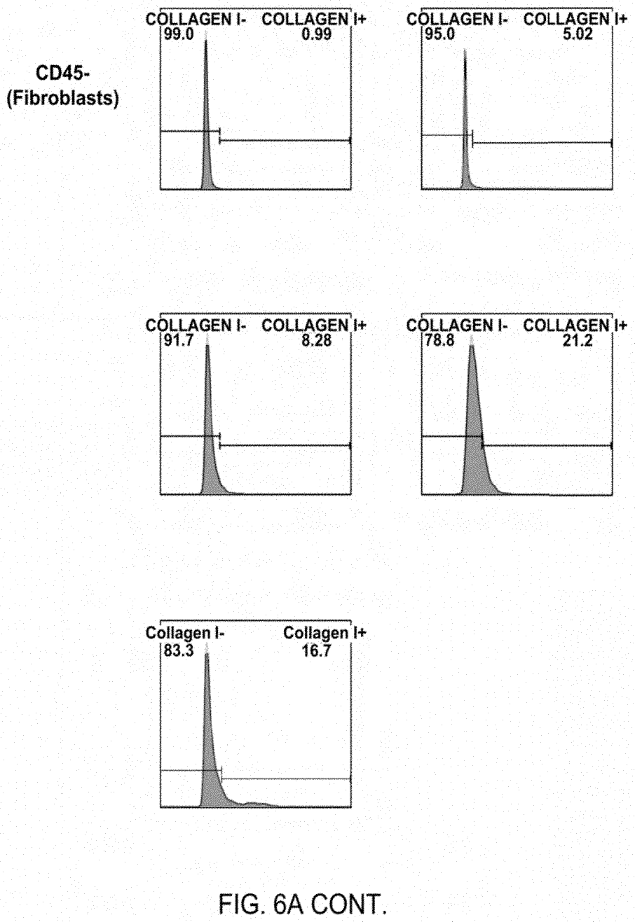

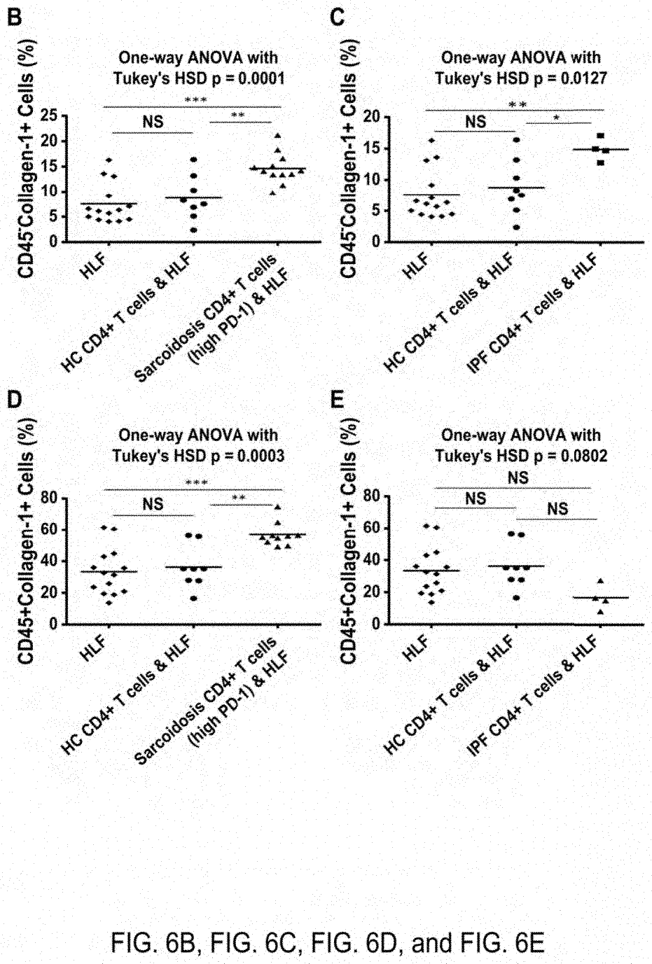

The percentage of PD-1+CD4+ T cells increase as pulmonary sarcoidosis progresses from granulomatous inflammation to fibrosis. Further investigation of humans and mice with pulmonary fibrosis, revealed that Th17 cells are the CD4+ T cell subset primarily responsible for TGF-β1 secretion in all three models. In order to determine for biologic and physiological significance of the observed TGF-β1 secretion, collagen I production was assessed following co-culture of human lung fibroblasts with sarcoidosis and IPF CD4+ T cells. To address this supposition, healthy control, sarcoidosis and IPF CD4+ T cells were purified to 90-98% purity and cultured them with human lung fibroblast cell line. FIG. 6A depicts the gating strategy using flow cytometry. During the analysis, CD3+CD4+ T cells were gated out, then used CD45 negative cells to identify the fibroblast population and CD45 positive cells to distinguish the fibrocyte population (FIG. 6A).

It was noticed a significant increase in percent collagen being produced by fibroblasts (CD45⁻) that had been cultured in the presence of sarcoidosis and IPF CD4+ T cells compared to fibroblasts cultured alone or in combination with HC CD4+ T cells (sarcoidosis, p=0.0002; IPF, p=0.01) (FIG. 6B-C). Fibroblasts that had been cultured with HC CD4+ T cells showed no significant difference in collagen-1 production, when compared to fibroblasts alone (p=0.53) (FIG. 6B-C). Culturing of human lung fibrocytes with sarcoidosis CD4+ T cells also induced collagen production (42% increase) (p=0.0006), but no significant increase was noted following co-culture with IPF CD4+ T cells (59% decrease) (p=0.08) (FIG. 6D-E). No significant differences were seen between fibrocytes cultured alone and fibrocytes cultured in conjunction with HC CD4+ T cells (p=0.67) (FIG. 6D-E). Notably, PD-1+CD4+T with levels akin to healthy controls did not induce collagen-1 production in co-culture experiments with fibroblasts or fibrocytes. These co-culture experiments also revealed evidence of cross-talk between CD4+ T cells and HLF. Strikingly, co-culturing of sarcoidosis and IPF CD4+ T cells with human lung fibroblasts significantly increased PD-L1 expression on both fibroblasts and fibrocytes populations (p<0.0001, p=0.0002; respectively). PD-L2 expression was not significantly altered.

(9) CD4+ T Cell-Dependent Human Lung Fibroblast Collagen I Production Requires PD-1

The detection of increased collagen-1 production following co-culture of sarcoidosis PD-1+CD4+ cells supported an investigation of the effects of PD-1 pathway blockade on HLF collagen I production. Purified sarcoidosis CD4+ T cells at a final concentration of 2×10⁶/mL were cultured overnight in the presence of α-PD-1, α-PD-L1 and α-PD-L2. HLFs were added the following day at a 1:10 ratio (HLF:T cell). The percent of collagen I produced by fibroblasts (CD45⁻) cultured in the presence of sarcoidosis CD4+ T cells following PD-1 pathway blockade dropped from 13.5% (mean) pre-blockade to 7.5% (mean) post-blockade, analogous to the control group [fibroblasts without the presence of CD4+ T cells (mean 7.5%)], a significant reduction of 44% (p<0.0007) (FIG. 7A-B). Interestingly, fibroblast collagen I expression was only reduced in the cells that were cultured with sarcoidosis CD4+ T cells expressing high baseline PD-1 (p=0.0006) (FIG. 7C) but not in those that had been cultured with sarcoidosis T cells from patients that expressed low baseline PD-1 levels (p=0.25) (FIG. 7D). Similar results were also observed for collagen I producing fibrocytes (CD45⁺) that were cultured in the presence of sarcoidosis CD4+ T cells. There was a significant decline in collagen I production of 22% following PD-1 pathway blockade (p=0.0002) (FIG. 7E). Once again, these differences were only observed when HLF were cultured with sorted CD4+ T cells from sarcoidosis patients that had high baseline PD-1 expression on their CD4+ T cells (p=0.0002) (FIG. 7F) but not from patients with low PD-1 (p=0.10) (FIG. 7G). Collectively, these results also imply that PD-1+CD4+ T cells have the capacity to induce pulmonary fibrosis, through stimulation on production of collagen 1 from human lung fibroblasts.

(10) PD-1 Blockade Reduces IL-17A and TGF-β1 Expression in CD4+ T Cells

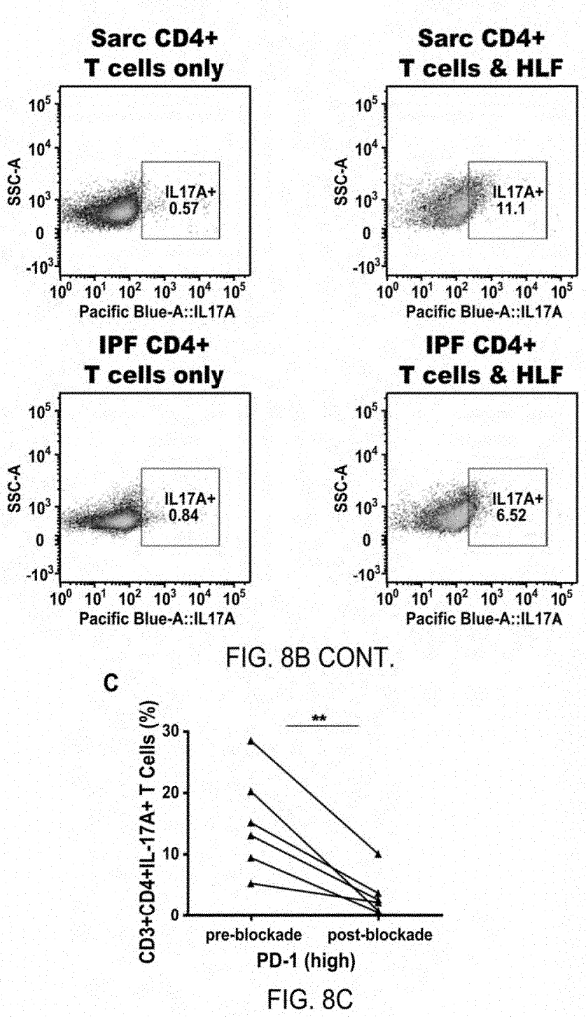

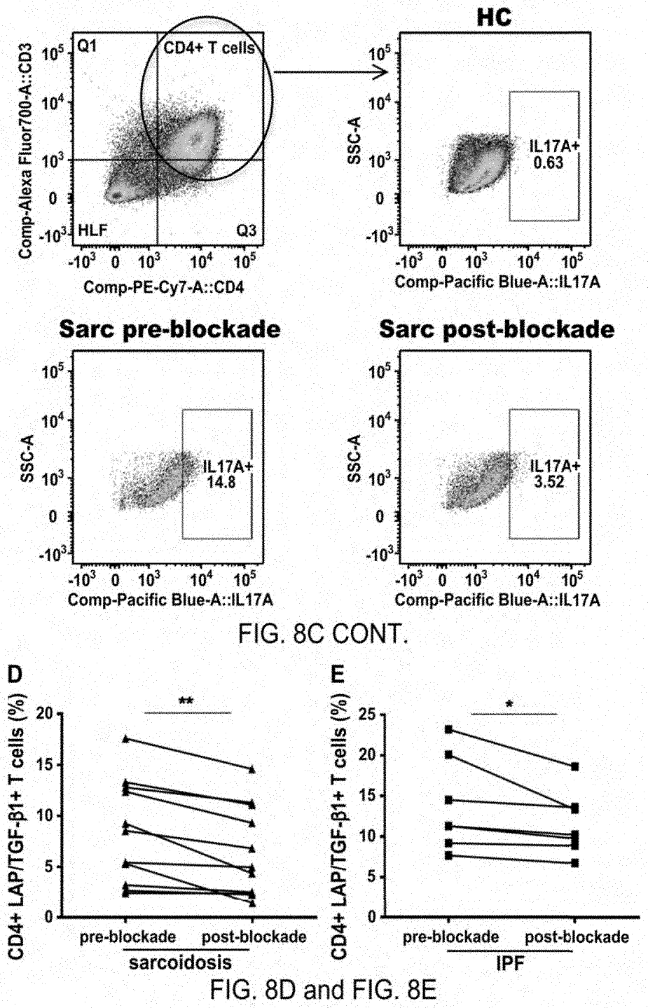

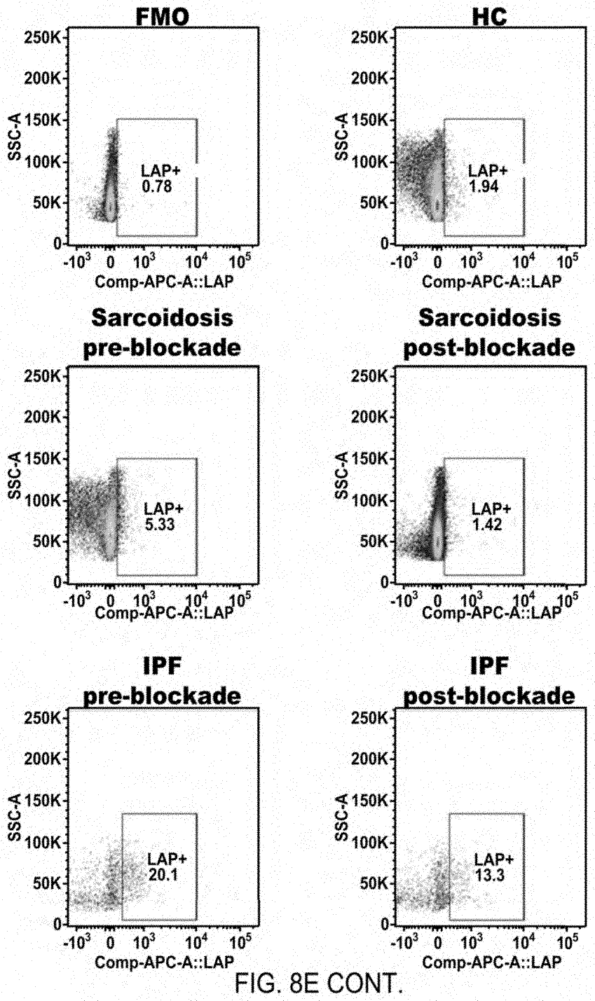

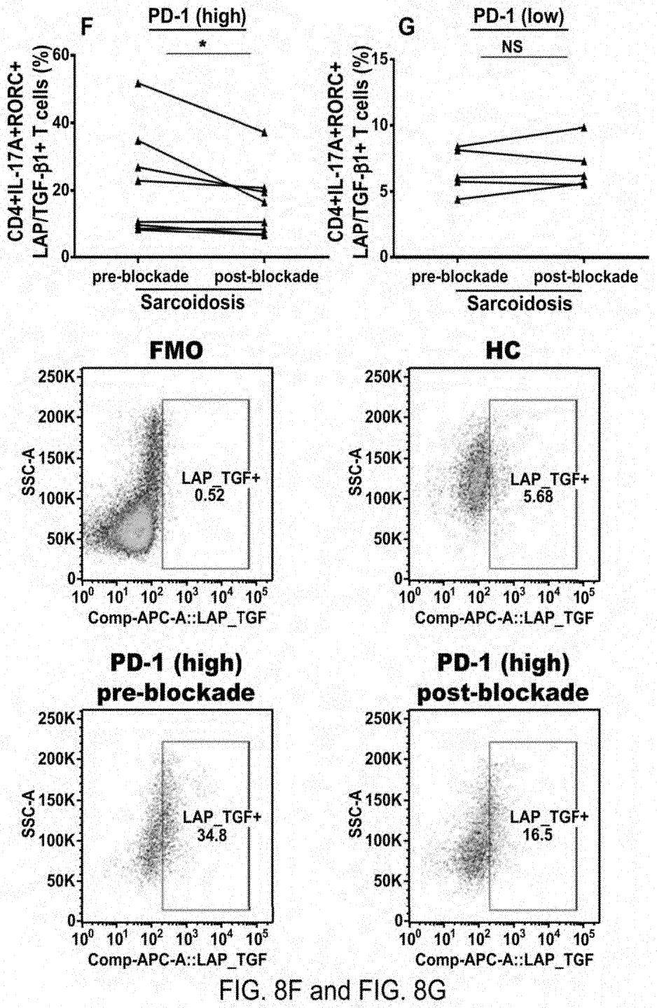

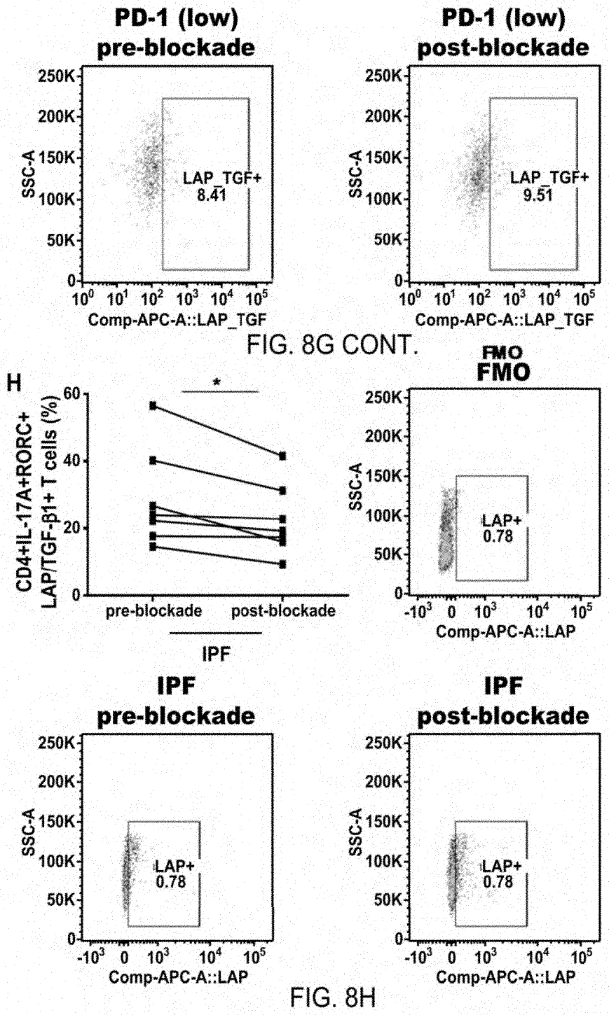

In order to identify how PD-1 was regulating collagen 1 production in HLF, for two key mediators of fibrosis were assessed: IL-17A and TGF-β. Co-culturing sarcoidosis CD4+ T cells with HLF resulted in a significant increase in IL-17A from CD4+ T cells (p=0.01) (FIG. 8A). HLF culturing with IPF CD4+ T cells also significantly increased IL-17A expression (p=0.0003) (FIG. 8B). The percent of IL-17A producing CD4+ T cells pre-blockade present after sarcoidosis CD4+ T cells were co-cultured with HLF was a mean of 15% (FIG. 8C). Following PD-1 pathway blockade, the percent of IL-17A producing CD4+ T cells in the co-culture experiments significantly declined to a mean of 3%, reflecting an 80% reduction (p=0.005) (FIG. 8C). The detection of increased IL-17A expression in the co-culture experiments supported further inquiry of the effects of the PD-1 pathway blockade on a key mediator of Th17 development, TGF-β, in both sarcoidosis and IPF CD4+ T cells. PD-1 pathway blockade significantly reduced TGF-β1 secretion on sarcoidosis CD4+ T cells (p=0.002) (FIG. 8D) and on IPF CD4+ T cells (p=0.04) (FIG. 8E). PD-1 pathway blockade effectively reduced TGF-β1 secretion from Th17 cells in the patients with high baseline PD-1 (p=0.04) (FIG. 8F) but not in subjects with PD-1+CD4+ T cell levels akin to healthy controls (p=0.49) (FIG. 8G). TGF-β1 expression was also significantly reduced in IPF Th17 cells also following blockade of PD-1 pathway (p=0.02) (FIG. 8H).

(11) PD-1 Blockade Reduces Expression of the Transcription Factor, STAT3, in CD4+ T Cells

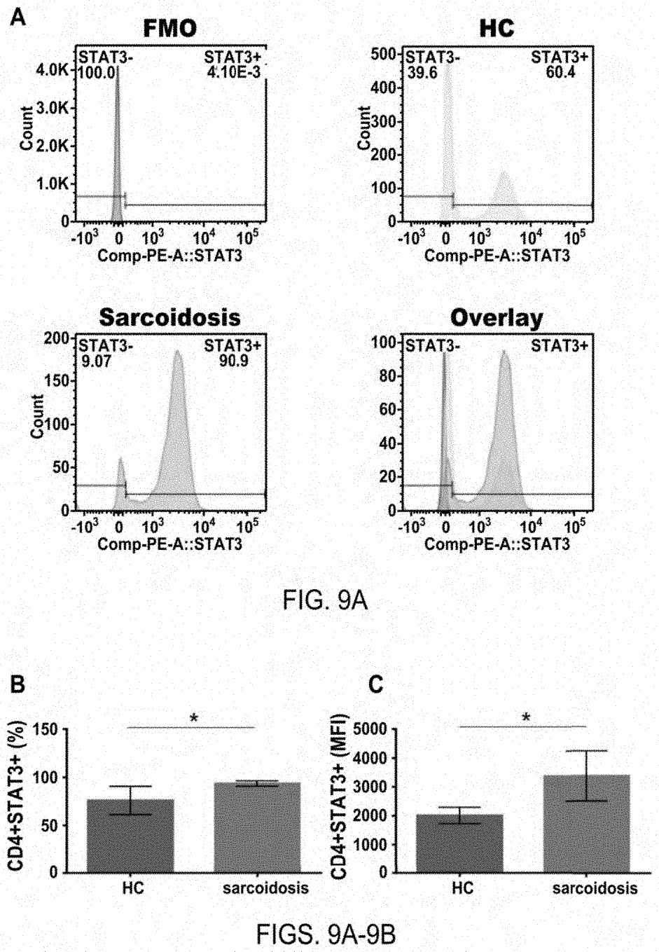

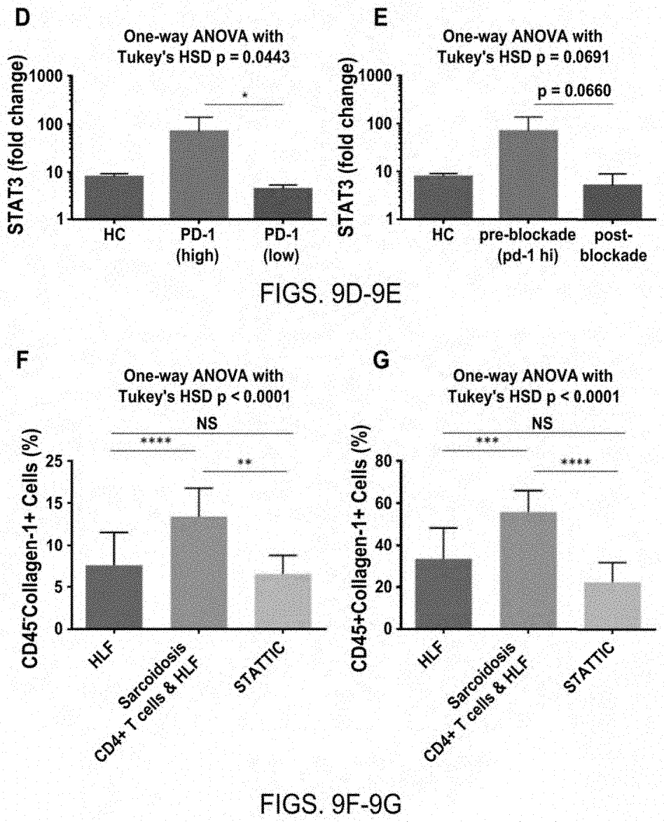

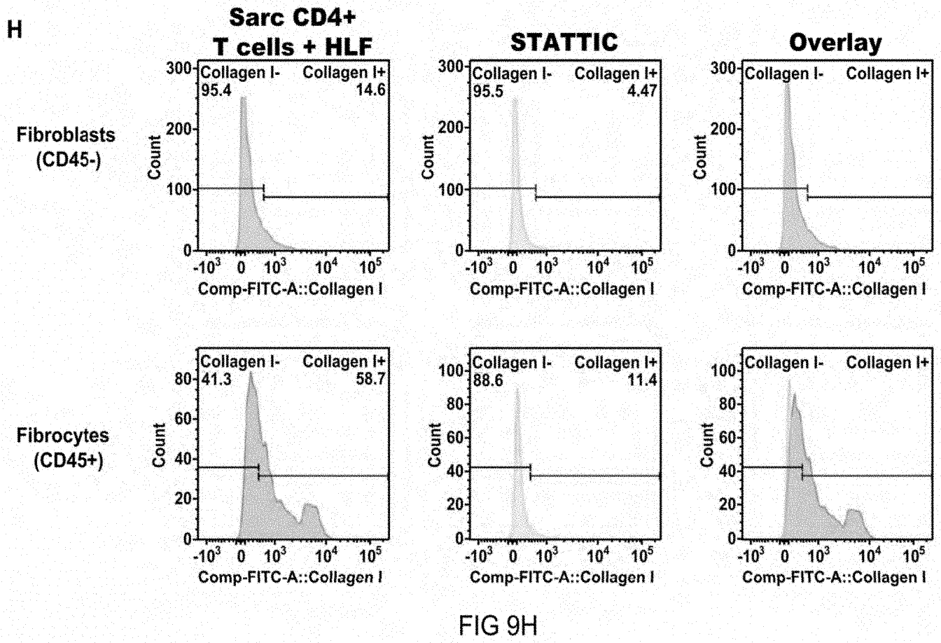

Th17 cells are IL-17A-producing CD4+ T cells that are vital in mucosal immunology. STAT3 is an essential regulator of Th17 cell development. In order to identify molecular mechanism by which PD-1 regulates IL-17A production, the critical transcription factor for Th17 development, STAT3 was the focus of investigation. Flow cytometric analysis for STAT3 expression revealed significantly higher expression in sarcoidosis subjects compared to controls. Distinctions in STAT3 MFI were also noted (FIG. 9A-C). Quantitative PCR analysis for STAT3 production demonstrated significant differences between sarcoidosis CD4+ T cells with high and lo PD-1 expression. Following PD-1 pathway blockade in sarcoidosis CD4+ T cells, reductions in STAT3 expression were noted (p=0.06)(FIG. 9D-E). In order to further confirm that PD-1 regulates collagen 1 production through STAT3-mediated IL-17 production, the small molecule inhibitor of STAT3, STATTIC was used. Sarcoidosis PD-1+CD4+ T cells that were co-cultured with human lung fibroblasts or fibrocytes resulted in a significant reduction in collagen 1 production to levels akin to HLF after exposure to STATTIC (FIG. 9 F-H).

(12) Elevated Expression of Th17-Related Pathway Genes is Associated with Reduced IPF Life Expectancy

Prior in vivo investigations demonstrate the importance of Th17 cells in pulmonary fibrosis. In order to further assess the relevancy of Th17 cells in interstitial lung diseases, IPF patients were screened for differences in gene expression profiles using Genome-Wide Association Studies (GWAS). Two distinct Th17 gene profiles were evident with significant differences in transplant-free survival in the discovery cohort (HR: 2.77, 95% CI: 0.96-8.01, p=0.042) (FIG. 10A). Similar to the immunologic investigation, enhanced TFS was present among IPF subjects possessing genetic signatures of reduced TGF-β, IL17A and increased IFNγ (FIG. 10A). Similar results were observed in the validation cohort where two cluster of patients were found to have significant differences in overall survival (HR: 5.24, 95% CI: 2.09-13.08, p=0.0001) (FIG. 10B). The hazard ratios (HR) for mortality and transplant-free survival ranged from 2.77 to 5.24, indicating an increased risk of dying or requiring a lung transplant for survival for patients with a genetic profile consistent with increased Th17 cell signaling (FIG. 10A-10B). These results suggest that dysregulation of Th17 genes is associated with IPF progression and poor disease outcome, providing further evidence of Th17 cell involvement in human interstitial lung disease.

b) Discussion

Key signaling mediators within the IL-17A pathway have been implicated in genetic investigations of both IPF and sarcoidosis; however, the immunologic significance of these findings was unclear. Here, it is that Programmed Death-1's regulation of TGFβ-1 and IL-17A expression in human and murine PD-1+CD4+ T cells carries biologic and clinical significance. It is shown that the IL-17A and TGFβ-1 produced from the PD-1+CD4+ T cells induce collagen-1 production upon co-culture with human lung fibroblasts. Furthermore, it was demonstrated that blockade of the PD-1 pathway significantly decreases IL-17A and TGFβ1 expression, with resultant declines in collagen-1 production, thus provides a new, readily available therapeutic for fibrotic lung disease patients. PD-1 regulation of STATS expression is a relevant mechanism to induce IL-17A induction of collagen 1 from HLF. The presented data now provides evidence of key signaling alterations following PD-1 upregulation that contribute to not only an immunosuppressive, but also a profibrotic, microenvironment in human and murine lungs.

There is an essential role for PD-1 upregulation in the creation of an immunosuppressive microenvironment in human interstitial lung disease. This work further expounds upon those observations by revealing that the immunosuppressive environment is characterized by increased PD-1+CD4+ cells with reduced proliferative capacity, and that the Th17 subsets contribute most significantly to TGF-β1 expression (FIG. 2, 5). The biologic significance of these Th17 cells are apparent in both GWAS and microarray analyses demonstrating increased morbidity and mortality with enhanced expression of genes along the Th17 pathway (Table 1, Table 2, FIG. 1). Strikingly, this immunologic significance is present in models of pulmonary fibrosis induced by epithelial injury (bleomycin), chronic antigenic stimulation from microbial antigens or genetic alterations in the SHP-2 signaling pathway (IPF). For example, intratracheal administration of bleomycin induces epithelial cell death and DNA destruction, whereas microbial antigens induce fibrosis through antigen-specific Th17 cells (Schistosomiasis). Th17 cells, producing both intracellular and membrane-bound TGF-β1, in human and murine models of pulmonary fibrosis were detected in sarcoidosis, IPF and bleomycin-induced pulmonary fibrosis (FIG. 5). Also noteworthy, was the observation that blockade of the PD-1 pathway resulted in reduced IL-17A and TGF-B1 production, with resultant diminution of collagen-1 production by HLF (FIG. 7). Together, these findings identify PD-1+Th17 cells as a critical mediator of pulmonary fibrosis, illustrating the potential utility of targeting the PD-1 pathway or IL-17A production in subjects with interstitial lung diseases.

Investigation of bleomycin-induced pulmonary fibrosis has revealed the essentiality of both TGFβ and IL-17A for collagen deposition. The presence of PD-1+CD4+ and CD8+ T cells upregulation in liver and skin fibrosis has been reported, but its role in interstitial lung disease has not been clearly delineated. While some studies have demonstrated the development of pulmonary fibrosis independent of TGF-β1, such as in the Schistosoma mansoni egg-induced pulmonary fibrosis, which is completely IL-13 dependent, the TGF-beta-1-CD44v6 pathway has been shown to be important in lung myofibroblast collagen 1 and alpha-SMA synthesis. Furthermore, TGF-beta-1-induced CD44v6 expression occurs through EGR1-mediated AP-1 (Activator protein-1) activity, and that the EGR1- and AP-1-binding sites in the CD44 promoter account for its responsiveness to TGF-beta-1 in lung fibroblasts. These elegant studies emphasize the appreciated important of the fibroblast in IPF pathogenesis. Here, it was demonstrated in in vitro and in vivo models that CD4+ T cells with PD-1 upregulation also carry biological significance. The observation that PD-1 blockade reduced TGF-β1 production in CD4+ T cells reveals the physiologic significance of PD-1 upregulation to lung fibrosis (FIG. 8). It was observed that reductions in TGF-β1 also were accompanied by decreased IL-17A expression, demonstrating that PD-1 provokes reductions in two direct mediators of pulmonary fibrosis (FIG. 8).

While independent investigations have identified important roles for TGF-β1 and IL-17A in pulmonary fibrosis, this investigation reveals that one mechanism by which PD-1 upregulation on CD4+ T cells drives fibrosis is through the STAT3 pathway (FIG. 8, 9). In liver fibrosis, global gene expression profiling revealed that the non-SMAD JAK1/STAT pathway is essential for the expression of a subset of TGF-β target genes, and that cooperation between the JAK1-STAT3 and SMAD pathways is critical to the roles of TGF-β in liver fibrosis. A significant reduction in TGF-β expression in CD4+ T cells after blockade of the PD-1 pathway (FIG. 9). While CD4+ T cells are not the only source of TGF-β, the identification of reduced collagen-1 production during co-cultured PD-1+CD4+ T cells with human fibroblasts following STAT3 inhibition further supports investigation of TGF-β/SMAD/STAT3 interactions in pulmonary fibrosis. In addition, STAT3 has a role in IL-17A and Th17 differentiation; it was noted significant reductions in IL-17A expression in CD4+ T cells following blockade of the PD-1 pathway (FIG. 8). The impact of chemical inhibition of the STAT3 pathway on collagen production reveals the biological significance of the STAT3 pathway and reveals another therapeutic target.

A limitation of this study is that other immunonologic and nonimmunologic contributors to lung fibrosis are not included. For example, mammalian sterile 20-like kinase 1 (MST1) signalling from DCs negatively regulates IL-17 producing-CD4⁺ T helper cell (Th17) differentiation. Future investigations that complement the understanding of extrinsic mediators of Th17 development and lung fibrosis are warranted. Increased PD-1 expression was noted on epithelial cells near fibroblastic foci using IHC (FIG. 4). The epithelial-mesenchymal transition (EMT) is a mechanism during which TGF-β1-induced molecular programming induces epithelial cells to develop phenotypic features associated with mesenchymal cells. The detection of increased expression of checkpoint inhibitors has not been appreciated. A role for checkpoint inhibitors in other diseases is being appreciated. Notably, in epithelial-derived malignancies, PD-L1 upregulation has been described and noted to promote EMT and accelerate carcinogenesis, thus indicating a close relationship between EMT and immune escape signaling pathways in cancer. Investigation of the role of checkpoint inhibitors on EMT in fibrotic lung disease is also warranted. Also, semaphorin 7A+T regulatory cells with reduced IL-10 expression have also been implicated in IPF progression.

In conclusion, blockade of the PD-1 pathway resulted in decreased STAT3 expression from sarcoidosis CD4+ T cells, resulting in significant declines in IL-17A and TGF-β expression that produced significant decliens collagen-1 production from HLF. The administration of PD-1 pathway blockade in bleomycin-induced pulmonary fibrosis also resulted in decreased fibrosis, thus providing in vivo confirmation of the relevance of PD-1 induction of pulmonary fibrosis. These data reveal immunologic overlap in fibrotic lung diseases regardless of etiology, as well as the relevance of PD-1 pathway to pulmonary fibrosis. Although rare, therapeutic targeting of the PD-1 pathway can induce lung infection, pneumonitis and interstitial lung disease. Judicious use of therapeutics of this pathway in patients with significant decline in pulmonary function should be carefully considered. Consideration of other readily available mediators of the STAT3 (metformin) which has been shown to alter EMT or antibody against IL-17A, which do not carry the same pulmonary adverse events, should be considered for the treatment of pulmonary fibrosis.

c) Materials and Methods

(1) Study Population

For study participation, clinical and radiographic criteria were used to define sarcoidosis as has been previously described. PBMC of patients from the University of Cincinnati, Cleveland Clinic and Vanderbilt University Medical Center were used. All subjects provided written informed consent that was approved by the appropriate Institutional Review Boards. All investigations with human subjects were conducted according to the principles expressed in the Helsinki Declaration. There were four subject cohorts: healthy controls (HC), subjects with sarcoidosis that have active disease, subjects with resolved disease and IPF patients. The sarcoidosis patients with active disease were characterized by reductions in FVC, radiographic progression, or pulmonary symptom acceleration. Sarcoidosis subjects who had resolved disease were distinguished by normalized FVC or chest radiograph and resolution of pulmonary symptoms despite not being on immunosuppressive therapy. Patients with high PD-1 expression were distinguished from patients with low PD-1 according to percent and mean fluorescent intensity (MFI). Subjects expressing low PD-1 were defined as having PD-1 percentages and MFIs akin to healthy controls. Study subject demographics are provided in Tables 1 and 2.

(2) PBMC Isolation and Freezing

Peripheral blood mononuclear cells (PBMC) were isolated from healthy control, sarcoidosis or IPF patient whole blood using Ficoll-Hypaque density gradient centrifugation. After PBMC collection, cells were frozen at 10×10⁶/mL in a 10% DMSO+FBS-containing cryotube. Samples were placed in a styrofoam container at −80° C. for no more than one week and then transferred to liquid nitrogen until use. Human lung fibroblasts/fibrocytes (HLF)-containing cryovials were shipped overnight from Sigma-Aldrich Inc. (Saint Louis, Mo.) on dry ice. HLF-containing cryovials were transferred to liquid nitrogen upon arrival until use. Experiments performed were limited to cellular availability.

(3) Microarray Analysis

The microarray gene expression dataset, GSE1907, was downloaded from the National Center for Biotechnology Information's Gene Expression Omnibus (GEO). Data were analyzed using Significant Analysis of Microarrays, as previously described. A stringent false-discovery rate cutoff of less than 1% was used to define statistical significance, using PCluster to depict microarrays analysis results.

(4) Immunohistochemistry

Diagnostic tissue blocks obtained from patients with sarcoidosis were provided by the Vanderbilt Translational Pathology Shared Resource with approval from the Vanderbilt Institutional Review Board. Negative control lung specimens were obtained from subjects who expired from nonpulmonary disease with no lung pathology Immunohistochemistry for PD-1 and PD-L1 were conducted as previously described, using anti-PD-L1 (clone 29E.2A3, BioLegend, San Diego, Calif.), and anti-PD-1 (clone 7A11B1, Sigma-Aldrich, St. Louis, Mo.). The Bond Refine Polymer detection system was used for visualization, after hematoxylin and eosin staining.

(5) Proliferation Assay

PBMC were labeled with carboxyfluorescein succinimidyl ester (CFSE) as previously described. Cells were then stimulated with plate-bound anti-CD3 (Ultra-LEAF Purified anti-human CD3, Biolegend, San Diego, Calif.) and soluble anti-CD28 antibodies (1 μg/mL, BD Biosciences, San Jose, Calif.) at a final concentration of 2×10⁶/mL in RPMI 1640-supplemented medium for 5 days at 37° C., 5% CO₂ atm.

(6) ELISA

CD4+ T cells were isolated from LN₂ frozen PBMC using Dynabeads CD4 Positive Isolation Kit (Invitrogen, Grand Island, N.Y.), according to the manufacturer's protocol. 2×10⁵ cells were plated in sets of triplicates per subject at a final concentration of 2×10⁶/mL in RPMI 1640-supplemented medium and incubated overnight at 37° C. in 5% atmospheric CO₂. Subsequent to overnight resting, the cells were TCR stimulated using plate-bound anti-CD3 (Ultra-LEAF Purified anti-human CD3, Biolegend, San Diego, Calif.) and soluble anti-CD28 (1 μg/mL, BD Biosciences, San Jose, Calif.) antibodies. Cell supernatants were collected succeeding a 48-hour incubation period with activation antibodies. Free activated TGF-β1 was determined from healthy control and sarcoidosis CD4+ T cells using the LEGEND MAX™ Free Active TGF-β1 ELISA Kit (Biolegend, San Diego, Calif.), as specified in the kit's instructions.

(7) CD4+ T Cell and Human Lung Fibroblast (HLF) Cell Co-Culture Experiments

LN₂ frozen PBMC were thawed and readied for CD4+ T cell purification as aforementioned. 4-5×10⁵ CD4+ T cells were plated on a 96-well plate at a final concentration of 2×10⁶/mL in RPMI 1640-supplemented medium and then allowed to rest at 37° C. in 5% atmospheric CO₂ for 2 hours prior to PD-1 pathway blockade or STATS inhibition using STATTIC. Subsequent to the 2 hours, CD4+ T cells were incubated overnight at 37° C. in 5% atmospheric CO₂ with or without the presence of anti-PD-1 (5 μg/mL), anti-PD-L1 (2 μg/mL) and anti-PD-L2 (2 μg/mL) blocking antibodies (Biolegend, San Diego, Calif.) or with STATTIC inhibitor. The following day, HLF were thawed using the fibroblast growth medium recommended by the manufacturer and following the Sigma-Aldrich Inc. (Saint Louis, Mo.) HLF thawing protocol. After thawing, HLF were suspended at 4×10⁶/mL in fibroblast growth medium and allowed to rest in a 37° C., 5% CO₂ humidified incubator. Isolated CD4+ T cells that had been incubated overnight with or without the presence of blockade antibodies were then TCR stimulated using plate-bound anti-CD3 (Ultra-LEAF Purified anti-human CD3, Biolegend, San Diego, Calif.) and soluble anti-CD28 (1 μg/mL, BD Biosciences, San Jose, Calif.) antibodies suspended in RPMI. HLF were then added to each condition at 1:10 (HLF:CD4+ T cell) as indicated by previous publications. For “HLF” only conditions, 200×10⁵ HLF were plated, final concentration of 2×10⁶/mL, in anti-CD3-coated wells and then soluble anti-CD28 in RPMI was added. Unstimulated conditions received the same treatment without the presence of TCR stimulation antibodies. All conditions were incubated for 4 days at 37° C. in 5% atmospheric CO² and stained with flow cytometry antibodies on the 5th day.

(8) Surface and Intracellular Flow Cytometry Antibodies

Anti-CD3 Alexa Fluor 700, CD4 PE-Cy7, PD-1 PerCP-Cy5.5, PD-1 BV 421, C274 APC, CD273 PE, RORC Alexa Fluor 488, TGF-β1 PE obtained from BD Biosciences (San Jose, Calif.), CD3 BV 785, LAP/TGF-β1 APC, CD45 BV 785, IL-17A BV421, IL-17A PE, CXCR5 APC-Cy7, CD25 PE-Cy5, FOXP3 Alexa Fluor 488, PD-1 BV 785, γδ TCR PerCP-Cy5.5, IFN-γ PerCP-Cy5.5 purchased from Biolegend (San Diego, Calif.) and collagen-1 FITC acquired from EMD Millipore Corp. (Billerica, Mass.) were used for human cell staining. For mice single-cell lung suspension staining, anti-CD3 Alexa Fluor 700, CD4 APC-Cy7, CXCR5 BV 785, IL-17A FITC, PD-1 PE-Cy7, FOXP3 Pacific Blue, LAP/TGF-β1 APC were purchased from Biolegend (San Diego, Calif.) and ROR-γt PE, IFN-γ PerCP-Cy5.5 were obtained from eBioscience (San Diego, Calif.). For mice single-cell lung suspension staining, anti-CD3 Alexa Fluor 700, CD4 APC-Cy7, CXCR5 BV 785, IL-17A FITC, PD-1 PE-Cy7, FOXP3 Pacific Blue, LAP/TGF-β1 APC were purchased from Biolegend (San Diego, Calif.) and ROR-γt PE, IFN-γ PerCP-Cy5.5 were obtained from eBioscience (San Diego, Calif.).

(9) Flow Cytometry Staining and Analysis

Subsequent to cell collection and washing, cells were incubated for 30 minutes in the dark at room temperature (RT) following the addition of surface staining flow cytometry antibodies. The cells were then fixed and permeabilized using the Intracellular Fixation and Permeabilization Buffer Set from eBiosciences (San Diego, Calif.) for 60 minutes in the dark at 4° C. Succeeding washing, cells were stained with intracellular antibodies for 45 minutes to 1 hour at RT in the dark. Fixed samples were placed at 4° C. in the dark for next day analysis. Data was acquired using BD LSRFortessa (BD Biosciences, San Jose, Calif.). Live cells and singlets were gated using forward and side scatter properties. Analysis was performed using FlowJo X software (Tree Star, Ashland, Oreg.). A minimum of 100,000 events were acquired per sample. Calibrator beads were used to calibrate the FACS machine for MFI acquisition.

(10) RNA Isolation and Quantitative RT-PCR

Total cellular RNA was extracted from purified CD4+ T cells following overnight incubation at 37° C. in 5% atmospheric CO₂ with (PD-1 blockade cohort) or without (pre-blockade cohort) the presence of anti-PD-1 (5 μg/mL), anti-PD-L1 (2 μg/mL) and anti-PD-L2 (2 μg/mL) blocking antibodies (Biolegend, San Diego, Calif.). Subsequent to overnight PD-1 pathway blockade, the cells were T cell receptor (TCR) stimulated using plate-bound CD3 and soluble CD28 antibodies for 48 hours as aforementioned. RNA extraction was performed according to the manufacturer's instructions (RNeasy Mini Kit, Qiagen, Germantown, Md.). After which, cDNA was generated as previously described. Quantitative RT-PCR amplification was performed in triplicates using 2× TaqMan Universal PCR Mastermix and TaqMan gene expression assays targeting STATS and GAPDH (Hs01047580_ml and Hs02758991_g1, Applied Biosystems/Life Technologies, Foster City, Calif.). Gene expression levels were normalized to β-actin and GAPDH. All reactions were carried out in a StepOncePlus Real Time PCR System (Applied Biosystems).

(11) Microarray Analyses

Peripheral blood gene expression data of two previously published IPF patient cohorts evaluated at the University of Chicago (N=45) (GSE27957) and the Imperial College London (N=55) (GSE93606) was analyzed. These cohorts were denominated discovery and replication cohort, respectively. To determine whether genes belonging to a TH17 signature were associated with transplant-free survival in IPF, in the discovery cohort, a hierarchical clustering algorithm was used that uses expression values of TH17 pre-selected genes, to cluster samples. To validate the results, hierarchical clustering was performed using matching probes of the TH17 signature in the discovery cohort.

(12) Animal Care and Bleomycin Experiments

All mouse experiments were approved by the Vanderbilt Institutional Animal Care and Use Committee (IACUC). Seven to nine week old C57BL/6J mice and PD-1 null obtained from the Jackson Laboratories were administered a single dose of bleomycin by intratracheal instillation as previously described. Lung specimens underwent H&E and trichrome staining. Hydroxyproline was measured by HPLC as previously described.

(13) Single Cell Suspensions

Following euthanasia of mice by CO₂ asphyxiation, lungs were perfused with 3 ml of ice-cold PBS to remove red blood cells. Lungs were then minced and incubated in collagenase 1 (0.7 mg/ml) in phenol-red free RPMI for 40 minutes at 37 C. Digests were then passed twice through a 70 μm Corning cell strainer to break any remaining aggregates, then centrifuged at 500×G for 5 minutes at 4° C. Red blood cells were lysed from the pellet. The resulting pellet was washed then resuspended in FACS buffer.

(14) Statistical Analysis

Comparisons between cohorts were performed using an unpaired two-tailed Student t test. Pre-blockade and PD-1 pathway blockade cohort comparisons were analyzed using a paired Student t test. Multiple group comparisons were performed using a one-way analysis of variance (ANOVA). Proliferation data were analyzed using the Mann-Whitney U test. Statistical analysis for all figures was carried out using Prism version 6.0 (GraphPad software). A P value <0.05 was considered statistically significant. All data was used in analysis.

### C. REFERENCES

A. Fischer et al., Am. J. Respir. Crit Care Med. 192, 727 (2015).
A. K. Shum et al., Sci. Transl. Med. 5, 206ra139 (2013).
A. M. Tager et al., Nat. Med. 14, 45 (2008).
A. O. Kamphorst et al., Science (2017).
A. Tzouvelekis et al., Am. J. Respir. Crit Care Med. 195, 500 (2017).
Adzhubei I, Jordan D M, Sunyaev S R. Predicting functional effect of human missense mutations using PolyPhen-2. Curr Protoc Hum Genet Editor Board Jonathan Haines Al. 2013 January; Chapter 7: Unit7.20. PMCID: PMC4480630
Benfaremo D, Manfredi L, Luchetti M M, Gabrielli A. Musculoskeletal and Rheumatic Diseases Induced by Immune Checkpoint Inhibitors: A Review of the Literature. Curr Drug Saf. 2018; 13(3):150-164. PMID: 29745339
Braun N A, Celada L J, Herazo-Maya J D, Abraham S, Shaginurova G, Sevin C M, Grutters J, Culver D A, Dworski R, Sheller J, Massion P P, Polosukhin V V, Johnson J E, Kaminski N, Wilkes D S, Oswald-Richter K A, Drake W P. Blockade of the programmed death-1 pathway restores sarcoidosis CD4(+) T-cell proliferative capacity. Am J Respir Crit Care Med. 2014 Sep. 1; 190(5):560-571. PMCID: PMC4214083
C. G. Lee et al., J. Exp. Med. 194, 809 (2001).
C. L. Maynard et al., J. Exp. Med. 206, 343 (2009).
C. Li et al., Nat. Commun. 8, 14275 (2017).
D. D. Patel, V. K. Kuchroo, Immunity. 43, 1040 (2015).
D. P. ten, H. M. Arthur, Nat. Rev. Mol. Cell Biol. 8, 857 (2007).
D. Pilling, T. Fan, D. Huang, B. Kaul, R. H. Gomer, PLoS. One. 4, e7475 (2009).
De Baets G, Van Durme J, Reumers J, Maurer-Stroh S, Vanhee P, Dopazo J, Schymkowitz J, Rousseau F. SNPeffect 4.0: on-line prediction of molecular and structural effects of protein-coding variants. Nucleic Acids Res. 2012 January; 40 (Database issue):D935-939. PMCID: PMC3245173
E. Bettelli et al., Nature 441, 235 (2006).
E. F. McKinney, J. C. Lee, D. R. Jayne, P. A. Lyons, K. G. Smith, Nature 523, 612 (2015).
E. Hams et al., Proc. Natl. Acad. Sci. U.S.A 111, 367 (2014).
E. Hui et al., Science (2017).
F. Gomez-Garcia et al., Br. J. Dermatol. 176, 594 (2017).
G. F. Sonnenberg et al., J. Exp. Med. 207, 1293 (2010).
Guo Y, Walsh A M, Canavan M, Wechalekar M D, Cole S, Yin X, Scott B, Loza M, Orr C, McGarry T, Bombardieri M, Humby F, Proudman S M, Pitzalis C, Smith M D, Friedman J R, Anderson I, Madakamutil L, Veale D J, Fearon U, Nagpal S Immune checkpoint inhibitor PD-1 pathway is down-regulated in synovium at various stages of rheumatoid arthritis disease progression. PloS One. 2018; 13(2):e0192704. PMCID: PMC5831027
H. M. Kang et al., Nat. Genet. 42, 348 (2010).
Hargadon K M, Johnson C E, Williams C J. Immune checkpoint blockade therapy for cancer: An overview of FDA-approved immune checkpoint inhibitors. Int Immunopharmacol. 2018 September; 62:29-39. PMID: 29990692
I. Gutcher et al., Immunity. 34, 396 (2011).
Immune Checkpoint Inhibitor (PD-1): Image Details—NCI Visuals Online [Internet]. [cited 2018 Oct. 22].
Immune-Related Adverse Events Associated with Immune Checkpoint Blockade|NEJM [Internet]. [cited 2018 Oct. 22].
J. D. Herazo-Maya et al., Sci. Transl. Med. 5, 205ra136 (2013).
J. E. Kolodsick et al., J. Immunol. 172, 4068 (2004).
J. J. O'Shea, W. E. Paul, Science 327, 1098 (2010).
J. Moisan, R. Grenningloh, E. Bettelli, M. Oukka, I. C. Ho, J. Exp. Med. 204, 2825 (2007).
J. T. Pesce et al., PLoS. Pathog. 5, e1000393 (2009).
J. Wahlstrom et al., J. Clin. Invest 117, 3576 (2007).
J. Wu, D. Hong, X. Zhang, X. Lu, J. Miao, Sci. Rep. 7, 44173 (2017).
Jerome R N, Pulley J M, Roden D M, Shirey-Rice J K, Bastarache L A, R Bernard G, B Ekstrom L, Lancaster W J, Denny J C. Using Human “Experiments of Nature” to Predict Drug Safety Issues: An Example with PCSK9 Inhibitors. Drug Saf. 2017 Nov. 28; PMID: 29185237
K. Cheng, M. Hao, Int. J. Mol. Sci. 17, (2016).
K. R. Kleaveland et al., J. Immunol. 193, 5229 (2014).
Kanehisa M, Sato Y, Kawashima M, Furumichi M, Tanabe M. KEGG as a reference resource for gene and protein annotation. Nucleic Acids Res. 2016 Jan. 4; 44(D1):D457-462. PMCID: PMC4702792
Karczewski K. The genome Aggregation Database (gnomAD) [Internet]. MacArthur Lab. 2017 [cited 2017 Mar. 18].
KEGG T01001: 5133 [Internet].
L. Ulloa, J. Doody, J. Massague, Nature 397, 710 (1999).
L. Y. Tang et al., J. Biol. Chem. 292, 4302 (2017).
LaFleur M W, Muroyama Y, Drake C G, Sharpe A H. Inhibitors of the PD-1 Pathway in Tumor Therapy. J Immunol Baltim Md 1950. 2018 Jan. 15; 200(2):375-383. PMCID: PMC5924692
M. H. Branton, J. B. Kopp, Microbes. Infect. 1, 1349 (1999).
M. S. Wilson et al., J. Exp. Med. 207, 535 (2010).
M. Veldhoen, R. J. Hocking, C. J. Atkins, R. M. Locksley, B. Stockinger, Immunity. 24, 179 (2006).
N. A. Braun et al., Am. J. Respir. Crit Care Med. 190, 560 (2014).
P. F. Piguet, M. A. Collart, G. E. Grau, A. P. Sappino, P. Vassalli, Nature 344, 245 (1990).
P. R. Mangan et al., Nature 441, 231 (2006).
Prokunina L, Castillejo-Lopez C, Oberg F, Gunnarsson I, Berg L, Magnusson V, Brookes A J, Tentler D, Kristjansdottir H, Grondal G, Bolstad A I, Svenungsson E, Lundberg I, Sturfelt G, Jonssen A, Truedsson L, Lima G, Alcocer-Varela J, Jonsson R, Gyllensten U B, Harley J B, Alarcon-Segovia D, Steinsson K, Alarcon-Riquelme M E. A regulatory polymorphism in PDCD1 is associated with susceptibility to systemic lupus erythematosus in humans. Nat Genet. 2002 December; 32(4):666-669. PMID: 12402038
R. A. Reilkoff et al., Am. J. Respir. Crit Care Med. 187, 180 (2013).
R. S. Lo et al., Am. J. Respir. Crit Care Med. 184, 1270 (2011).
RCSB PDB: Homepage [Internet].
S. A. Khader, S. L. Gaffen, J. K. Kolls, Mucosal. Immunol. 2, 403 (2009).
S. Ghatak et al., J. Biol. Chem. (2017).
S. Ghatak et al., J. Biol. Chem. 289, 7856 (2014).
S. I. Chung et al., Sci. Rep. 6, 39714 (2016).
S. Zhou et al., PLoS. Negl. Trop. Dis. 10, e0005094 (2016).
T. E. King, Jr. et al., N. Engl. J. Med. 370, 2083 (2014).
T. Kudo et al., Lancet Oncol. (2017).
T. Okazaki et al., J. Immunol. 167, 1977 (2001).
UniProt Consortium. UniProt: a hub for protein information. Nucleic Acids Res. 2015 January; 43(Database issue):D204-212. PMCID: PMC4384041
Wieder T, Eigentler T, Brenner E, Rocken M. Immune checkpoint blockade therapy. J Allergy Clin Immunol. 2018 Mar. 27; PMID: 29596939
Wu C, Macleod I, Su A I. BioGPS and MyGene.info: organizing online, gene-centric information. Nucleic Acids Res. 2013 January; 41(Database issue):D561-565. PMCID: PMC3531157
X. M. Meng, D. J. Nikolic-Paterson, H. Y. Lan, Nat. Rev. Nephrol. 12, 325 (2016).
X. O. Yang et al., J. Biol. Chem. 282, 9358 (2007).
Y. Cao et al., Cancer Res. 71, 1235 (2011).
Z. Song et al., J. Exp. Med. 201, 755 (2005).

1. A method of treating pulmonary disease or collagen-1 production in a subject with a pulmonary disease comprising administering to the subject an agent that reduces STAT3 production or activation.

2. The method of claim 1, wherein the pulmonary disease is an interstitial lung disease.

3. The method of claim 2, wherein the interstitial lung disease comprises idiopathic pulmonary fibrosis, bleomycin-induced pulmonary fibrosis, or sarcoidosis.

4. The method of claim 1, wherein the agent is a small molecule, peptide, polypeptide, siRNA, or antibody.

5. The method of claim 4, wherein in the agent is an antibody.

6. The method of claim 1, wherein the agent inhibits the interaction of PD-1 and PDL-1.

7. The method of any of claim 6, wherein the agent is an anti-PD1 antibody.

8. The method of claim 1, wherein the agent inhibits the proliferation of T_{H}17 cells.

9. The method of claim 1, wherein the agent comprises a STAT3 inhibitor.

10. The method of claim 9, wherein the STAT3 inhibitor is selected from the group consisting of Tyrosine phosphorylation inhibitors, SH2 domain inhibitors or dimerizaton inhibitors, and DNA binding domain inhibitors.

11. A method of creating a prognosis for a subject with a pulmonary disease comprising obtaining a tissue sample from a subject, isolating the TGFβ+PD1+CD4 T cells (T_{H}17 cells), measuring the number of T_{H}17 CD4+ T cells or the IL-17 expression levels relative to a control, wherein an increase in the number of T_{H}17 cells or an increase in IL-17 expression levels relative to a control indicates a more aggressive disease and decreased survival chances for the subject.

12. The method of claim 11, wherein the T_{H}17 expression is measured by microarray, flow cytometry, or ELISA.

13. (canceled)

14. (canceled)

15. A method of creating a prognosis for a subject with a pulmonary disease comprising obtaining a tissue sample from a subject, isolating the TGFβ+PD1+CD4 T cells (T_{H}17 cells), assaying for single nucleotide polymorphisms (SNPs) in the STAT3 gene, IL-17RA gene, IL-17D gene, ERAP2 gene, PDCD1 gene, and/or TGF-β1 gene, wherein the presence of an assayed SNP in the STAT3 gene, IL-17RA gene, PDCD1 gene, or TGF-β1 gene indicate an increased risk of disease progression, the presence of a assayed SNP in the IL-17D gene indicates increased risk of persistence, and the presence of an assayed SNP in the ERAP2 gene indicates increased risk of disease progression and persistence.

16. The method of claim 15, wherein the SNP is identified by allele-specific PCR, sequencing, single base extension (SBE), oligonucleotide ligation assay (OLA), Heated oligonucleotide ligation assay (HOLA), mass spectrometry, single-strand conformation polymophism, and/or electrophoresis.

17. A method of treating pulmonary disease in a subject comprising administering to the subject an inhibitor IL-17A and/or TGFβ-1.

18. (canceled)
